SEQUENCE LISTING (96 sequences)

SEQ ID NO: 1 - protein, 16 aa, Artificial Sequence
  Description of Artificial Sequence Synthetic peptide
RSSQSLVHRNGNTYLH

SEQ ID NO: 2 - protein, 7 aa, Artificial Sequence
  Description of Artificial Sequence Synthetic peptide
TVSNRFS

SEQ ID NO: 3 - protein, 9 aa, Artificial Sequence
  Description of Artificial Sequence Synthetic peptide
SQSSHVPPT

SEQ ID NO: 4 - protein, 5 aa, Artificial Sequence
  Description of Artificial Sequence Synthetic peptide
NYGVN

SEQ ID NO: 5 - protein, 17 aa, Artificial Sequence
  Description of Artificial Sequence Synthetic peptide
WINPNTGEPTFDDDFKG

SEQ ID NO: 6 - protein, 11 aa, Artificial Sequence
  Description of Artificial Sequence Synthetic peptide
SRGKNEAWFAY

SEQ ID NO: 7 - protein, 5 aa, Artificial Sequence
  Description of Artificial Sequence Synthetic peptide
NYGMN

SEQ ID NO: 8 - protein, 17 aa, Artificial Sequence
  Description of Artificial Sequence Synthetic peptide
WINTYTREPTYADDFKG

SEQ ID NO: 9 - protein, 12 aa, Artificial Sequence
  Description of Artificial Sequence Synthetic peptide
DITAVVPTGFDY

SEQ ID NO: 10 - protein, 11 aa, Artificial Sequence
  Description of Artificial Sequence Synthetic peptide
RASENIYSNLA

SEQ ID NO: 11 - protein, 7 aa, Artificial Sequence
  Description of Artificial Sequence Synthetic peptide
AASNLAD

SEQ ID NO: 12 - protein, 9 aa, Artificial Sequence
  Description of Artificial Sequence Synthetic peptide
QHFWTTPWA

SEQ ID NO: 13 - DNA, 21 bp, Artificial Sequence
  Description of Artificial Sequence Synthetic oligonucleotide
aatgcggcggtggtgacagta

SEQ ID NO: 14 - DNA, 21 bp, Artificial Sequence
  Description of Artificial Sequence Synthetic oligonucleotide
aagctcagcacacagaaagac

SEQ ID NO: 15 - DNA, 21 bp, Artificial Sequence
  Description of Artificial Sequence Synthetic oligonucleotide
uaaaaucuuccugcccacctt

SEQ ID NO: 16 - DNA, 21 bp, Artificial Sequence
  Description of Artificial Sequence Synthetic oligonucleotide
ggaagcuguuggcugaaaatt

SEQ ID NO: 17 - RNA, 21 nt, Artificial Sequence
  Description of Artificial Sequence Synthetic oligonucleotide
aagaccagccucuuugcccag

SEQ ID NO: 18 - DNA, 19 bp, Artificial Sequence
  Description of Artificial Sequence Synthetic oligonucleotide
ggaccaggcagaaaacgag

SEQ ID NO: 19 - RNA, 17 nt, Artificial Sequence
  Description of Artificial Sequence Synthetic oligonucleotide
cuaucaggaugacgcgg

SEQ ID NO: 20 - RNA, 21 nt, Artificial Sequence
  Description of Artificial Sequence Synthetic oligonucleotide
ugacacaggcaggcuugacuu

SEQ ID NO: 21 - DNA, 19 bp, Artificial Sequence
  Description of Artificial Sequence Synthetic oligonucleotide
ggtgaagaagggcgtccaa

SEQ ID NO: 22 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 23 - DNA, 20 bp, Artificial Sequence
  Description of Artificial Sequence Synthetic oligonucleotide
aggtggtgttaacagcagag

SEQ ID NO: 24 - DNA, 21 bp, Artificial Sequence
  Description of Artificial Sequence Synthetic oligonucleotide
aaggtggagcaagcggtggag

SEQ ID NO: 25 - DNA, 21 bp, Artificial Sequence
  Description of Artificial Sequence Synthetic oligonucleotide
aaggagttgaaggccgacaaa

SEQ ID NO: 26 - DNA, 21 bp, Artificial Sequence
  Description of Artificial Sequence Synthetic oligonucleotide
uauggagcugcagaggaugtt

SEQ ID NO: 27 - DNA, 49 bp, Artificial Sequence
  Description of Artificial Sequence Synthetic oligonucleotide
tttgaatatctgtgctgagaacacagttctcagcacagatattcttttt

SEQ ID NO: 28 - DNA, 29 bp, Artificial Sequence
  Description of Artificial Sequence Synthetic oligonucleotide
aatgagaaaagcaaaaggtgccctgtctc

SEQ ID NO: 29 - RNA, 21 nt, Artificial Sequence
  Description of Artificial Sequence Synthetic oligonucleotide
aaucaucaucaagaaagggca

SEQ ID NO: 30 - DNA, 21 bp, Artificial Sequence
  Description of Artificial Sequence Synthetic oligonucleotide
augacugucaggauguugctt

SEQ ID NO: 31 - RNA, 21 nt, Artificial Sequence
  Description of Artificial Sequence Synthetic oligonucleotide
gaacgaauccugaagacaucu

SEQ ID NO: 32 - DNA, 29 bp, Artificial Sequence
  Description of Artificial Sequence Synthetic oligonucleotide
aagcctggctacagcaatatgcctgtctc

SEQ ID NO: 33 - DNA, 21 bp, Artificial Sequence
  Description of Artificial Sequence Synthetic oligonucleotide
ugaccaucaccgaguuuautt

SEQ ID NO: 34 - DNA, 21 bp, Artificial Sequence
  Description of Artificial Sequence Synthetic oligonucleotide
aagtcggacgcaacagagaaa

SEQ ID NO: 35 - DNA, 21 bp, Artificial Sequence
  Description of Artificial Sequence Synthetic oligonucleotide
cuaccuuucuacggacgugtt

SEQ ID NO: 36 - DNA, 21 bp, Artificial Sequence
  Description of Artificial Sequence Synthetic oligonucleotide
ctgcctaaggcggatttgaat

SEQ ID NO: 37 - DNA, 21 bp, Artificial Sequence
  Description of Artificial Sequence Synthetic oligonucleotide
ttauuccuucuucgggaaguc

SEQ ID NO: 38 - DNA, 21 bp, Artificial Sequence
  Description of Artificial Sequence Synthetic oligonucleotide
aaccttctggaacccgcccac

SEQ ID NO: 39 - DNA, 19 bp, Artificial Sequence
  Description of Artificial Sequence Synthetic oligonucleotide
gagcatcttcgagcaagaa

SEQ ID NO: 40 - DNA, 19 bp, Artificial Sequence
  Description of Artificial Sequence Synthetic oligonucleotide
catgtggcaccgtttgcct

SEQ ID NO: 41 - DNA, 21 bp, Artificial Sequence
  Description of Artificial Sequence Synthetic oligonucleotide
aactaccagaaaggtatacct

SEQ ID NO: 42 - DNA, 21 bp, Artificial Sequence
  Description of Artificial Sequence Synthetic oligonucleotide
ucacaguguccuuuauguatt

SEQ ID NO: 43 - DNA, 21 bp, Artificial Sequence
  Description of Artificial Sequence Synthetic oligonucleotide
gcaugaaccggaggcccautt

SEQ ID NO: 44 - DNA, 19 bp, Artificial Sequence
  Description of Artificial Sequence Synthetic oligonucleotide
ccggacagttccatgtata

SEQ ID NO: 45 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 46 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 47 - protein, 17 aa, Artificial Sequence
  Description of Artificial Sequence Synthetic peptide
QIEYLAKQIVDNAIQQA

SEQ ID NO: 48 - protein, 21 aa, Artificial Sequence
  Description of Artificial Sequence Synthetic peptide
CGQIEYLAKQIVDNAIQQAGC

SEQ ID NO: 49 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 50 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 51 - protein, 23 aa, Artificial Sequence
  Description of Artificial Sequence Synthetic peptide
CGFEELAWKIAKMIWSDVFQQGC

SEQ ID NO: 52 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 53 - protein, 29 aa, Artificial Sequence
  Description of Artificial Sequence Synthetic peptide
GSGGGGSGGGGSQIEYLAKQIVDNAIQQA

SEQ ID NO: 54 - protein, 51 aa, Homo sapiens
SLRECELYVQKHNIQALLKDVSIVQLCTARPERPMAFLREYFEKLEKEEAK

SEQ ID NO: 55 - protein, 54 aa, Homo sapiens
SLKGCELYVQLHGIQQVLKDCIVHLCISKPERPMKFLREHFEKLEKEENRQILA

SEQ ID NO: 56 - protein, 44 aa, Homo sapiens
SHIQIPPGLTELLQGYTVEVGQQPPDLVDFAVEYFTRLREARRQ

SEQ ID NO: 57 - protein, 44 aa, Homo sapiens
SIEIPAGLTELLQGFTVEVLRHQPADLLEFALQHFTRLQQENER

SEQ ID NO: 58 - protein, 17 aa, Artificial Sequence
  Description of Artificial Sequence Synthetic peptide
QIEYVAKQIVDYAIHQA

SEQ ID NO: 59 - protein, 17 aa, Artificial Sequence
  Description of Artificial Sequence Synthetic peptide
QIEYKAKQIVDHAIHQA

SEQ ID NO: 60 - protein, 17 aa, Artificial Sequence
  Description of Artificial Sequence Synthetic peptide
QIEYHAKQIVDHAIHQA

SEQ ID NO: 61 - protein, 17 aa, Artificial Sequence
  Description of Artificial Sequence Synthetic peptide
QIEYVAKQIVDHAIHQA

SEQ ID NO: 62 - protein, 18 aa, Artificial Sequence
  Description of Artificial Sequence Synthetic peptide
PLEYQAGLLVQNAIQQAI

SEQ ID NO: 63 - protein, 18 aa, Artificial Sequence
  Description of Artificial Sequence Synthetic peptide
LLIETASSLVKNAIQLSI

SEQ ID NO: 64 - protein, 18 aa, Artificial Sequence
  Description of Artificial Sequence Synthetic peptide
LIEEAASRIVDAVIEQVK

SEQ ID NO: 65 - protein, 18 aa, Artificial Sequence
  Description of Artificial Sequence Synthetic peptide
ALYQFADRFSELVISEAL

SEQ ID NO: 66 - protein, 17 aa, Artificial Sequence
  Description of Artificial Sequence Synthetic peptide
LEQVANQLADQIIKEAT

SEQ ID NO: 67 - protein, 17 aa, Artificial Sequence
  Description of Artificial Sequence Synthetic peptide
FEELAWKIAKMIWSDVF

SEQ ID NO: 68 - protein, 18 aa, Artificial Sequence
  Description of Artificial Sequence Synthetic peptide
ELVRLSKRLVENAVLKAV

SEQ ID NO: 69 - protein, 18 aa, Artificial Sequence
  Description of Artificial Sequence Synthetic peptide
TAEEVSARIVQVVTAEAV

SEQ ID NO: 70 - protein, 18 aa, Artificial Sequence
  Description of Artificial Sequence Synthetic peptide
QIKQAAFQLISQVILEAT

SEQ ID NO: 71 - protein, 16 aa, Artificial Sequence
  Description of Artificial Sequence Synthetic peptide
LAWKIAKMIVSDVMQQ

SEQ ID NO: 72 - protein, 24 aa, Artificial Sequence
  Description of Artificial Sequence Synthetic peptide
DLIEEAASRIVDAVIEQVKAAGAY

SEQ ID NO: 73 - protein, 18 aa, Artificial Sequence
  Description of Artificial Sequence Synthetic peptide
LEQYANQLADQIIKEATE

SEQ ID NO: 74 - protein, 20 aa, Artificial Sequence
  Description of Artificial Sequence Synthetic peptide
FEELAWKIAKMIWSDVFQQC

SEQ ID NO: 75 - protein, 17 aa, Artificial Sequence
  Description of Artificial Sequence Synthetic peptide
QIEYLAKQIPDNAIQQA

SEQ ID NO: 76 - protein, 25 aa, Artificial Sequence
  Description of Artificial Sequence Synthetic peptide
KGADLIEEAASRIVDAVIEQVKAAG

SEQ ID NO: 77 - protein, 25 aa, Artificial Sequence
  Description of Artificial Sequence Synthetic peptide
KGADLIEEAASRIPDAPIEQVKAAG

SEQ ID NO: 78 - protein, 25 aa, Artificial Sequence
  Description of Artificial Sequence Synthetic peptide
PEDAELVRLSKRLVENAVLKAVQQY

SEQ ID NO: 79 - protein, 25 aa, Artificial Sequence
  Description of Artificial Sequence Synthetic peptide
PEDAELVRTSKRLVENAVLKAVQQY

SEQ ID NO: 80 - protein, 25 aa, Artificial Sequence
  Description of Artificial Sequence Synthetic peptide
PEDAELVRLSKRDVENAVLKAVQQY

SEQ ID NO: 81 - protein, 25 aa, Artificial Sequence
  Description of Artificial Sequence Synthetic peptide
PEDAELVRLSKRLPENAVLKAVQQY

SEQ ID NO: 82 - protein, 25 aa, Artificial Sequence
  Description of Artificial Sequence Synthetic peptide
PEDAELVRLSKRLPENAPLKAVQQY

SEQ ID NO: 83 - protein, 25 aa, Artificial Sequence
  Description of Artificial Sequence Synthetic peptide
PEDAELVRLSKRLVENAVEKAVQQY

SEQ ID NO: 84 - protein, 25 aa, Artificial Sequence
  Description of Artificial Sequence Synthetic peptide
EEGLDRNEEIKRAAFQIISQVISEA

SEQ ID NO: 85 - protein, 25 aa, Artificial Sequence
  Description of Artificial Sequence Synthetic peptide
LVDDPLEYQAGLLVQNAIQQAIAEQ

SEQ ID NO: 86 - protein, 25 aa, Artificial Sequence
  Description of Artificial Sequence Synthetic peptide
QYETLLIETASSLVKNAIQLSIEQL

SEQ ID NO: 87 - protein, 25 aa, Artificial Sequence
  Description of Artificial Sequence Synthetic peptide
LEKQYQEQLEEEVAKVIVSMSIAFA

SEQ ID NO: 88 - protein, 25 aa, Artificial Sequence
  Description of Artificial Sequence Synthetic peptide
NTDEAQEELAWKIAKMIVSDIMQQA

SEQ ID NO: 89 - protein, 25 aa, Artificial Sequence
  Description of Artificial Sequence Synthetic peptide
VNLDKKAVLAEKIVAEAIEKAEREL

SEQ ID NO: 90 - protein, 25 aa, Artificial Sequence
  Description of Artificial Sequence Synthetic peptide
NGILELETKSSKLVQNIIQTAVDQF

SEQ ID NO: 91 - protein, 25 aa, Artificial Sequence
  Description of Artificial Sequence Synthetic peptide
TQDKNYEDELTQVALALVEDVINYA

SEQ ID NO: 92 - protein, 25 aa, Artificial Sequence
  Description of Artificial Sequence Synthetic peptide
ETSAKDNINIEEAARFLVEKILVNH

SEQ ID NO: 93 - protein, 21 aa, Artificial Sequence
  Description of Artificial Sequence Synthetic peptide
CGQIEYLAKQIVDNAIQQAGC

SEQ ID NO: 94 - protein, 22 aa, Artificial Sequence
  Description of Artificial Sequence Synthetic peptide
RSQSRSRYYRQRQRSRRRRRRS

SEQ ID NO: 95 - DNA, 21 bp, Artificial Sequence
  Description of Artificial Sequence Synthetic oligonucleotide
ccggacaguuccauguauatt

SEQ ID NO: 96 - protein, 15 aa, Artificial Sequence
  Description of Artificial Sequence Synthetic peptide
GGGGSGGGGSGGGGS

(19) United States
(12) Patent Application Publication
(10) Pub. No.: US 2012/0321556 A1
(43) Pub. Date: Dec. 20, 2012

(54) Enhanced Cytotoxicity of Anti-CD74 and Anti-HLA-DR Antibodies with Interferon-Gamma

(75) Inventors: Jack D. Burton, Long Island City, NY (US); Rhona Stein, Maplewood, NJ (US); David M. Goldenberg, Mendham, NJ (US)

(73) Assignees: IMMUNOMEDICS, INC., Morris Plains, NJ (US); CENTER FOR MOLECULAR MEDICINE AND IMMUNOLOGY, Morris Plains, NJ (US)

(21) Appl. No.: 13/567,226

(22) Filed: Aug. 6, 2012

Related U.S. Application Data
(62) Division of application No. 13/004,349, filed on Jan. 11, 2011.
(60) Provisional application No. 61/293,846, filed on Jan. 11, 2010.
(60) Provisional application No. 61/323,001, filed on Apr. 12, 2010.
(60) Provisional application No. 61/374,449, filed on Aug. 17, 2010.

(51) Int. Cl. A61K 39/395; A61P 35/02; A61P 37/00; A61P 37/06; A61K 38/20; A61P 19/02; A61P 1/04; A61P 1/16; A61K 51/10; A61K 38/19; A61P 35/00; A61P 3/10

(52) U.S. Cl. 424/1.49; 424/172.1; 424/174.1; 424/133.1; 424/178.1; 424/136.1; 424/85.1; 424/85.2; 424/135.1

(57) ABSTRACT

Disclosed herein are methods and compositions comprising interferon-γ (IFN-γ) and anti-CD74 or anti-HLA-DR antibodies. In preferred embodiments, the IFN-γ increases the expression of CD74 and/or HLA-DR in target cells and increases the sensitivity of the cells to the cytotoxic effects of the anti-CD74 or anti-HLA-DR antibodies. The compositions and methods are of use to treat diseases involving CD74⁺ and/or HLA-DR⁺ cells, such as cancer cells, autoimmune disease cells or immune dysfunction disease cells.

### CROSS-REFERENCE TO RELATED APPLICATIONS

This application is a divisional of U.S. application Ser. No. 13/004,349, filed Jan. 11, 2011, which claims the benefit under 35 U.S.C. 119(e) of provisional application Ser. Nos. 61/293,846, filed Jan. 11, 2010; 61/323,001, filed Apr. 12, 2010; and 61/374,449, filed Aug. 17, 2010. Each of the priority applications is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Jan. 3, 2011, is named CMMI218U.txt and is 30,743 bytes in size.

### FIELD OF THE INVENTION

The present invention concerns compositions and methods of therapeutic treatment of cancer and/or other diseases involving CD74 positive cells. Preferably, the compositions and methods relate to use of interferon-gamma to increase expression of CD74 (also known as the invariant chain (Ii) of the HLA-DR complex) and to increase sensitivity of cancer cells to anti-CD74 antibodies, antibody fragments and/or immunoconjugates. In more preferred embodiments, the methods and compositions are effective to treat hematopoietic cancers, including but not limited to leukemias, lymphomas, non-Hodgkin's lymphoma (NHL), multiple myeloma, chronic lymphocytic leukemia, acute lymphocytic leukemia, acute myelogenous leukemia, glioblastoma, follicular lymphoma and diffuse large B cell lymphoma. However, the renal cancer, lung cancer, stomach cancer, breast cancer, prostate cancer, ovarian cancer and melanoma, express CD74 and any such cancer may be treated using the disclosed methods and compositions. The methods and compositions are also of use for other diseases associated with CD74 positive cells, such as autoimmune disease or immune dysregulation disease (e.g., graft-versus-host disease, organ transplant rejection). In alternative embodiments, the compositions and methods may involve use of interferon-gamma to increase expression of HLA-DR and enhance sensitivity of HLA-DR positive cells to anti-HLA-DR antibodies or fragments thereof.

### BACKGROUND

The human leukocyte antigen—DR(HLA-DR) is one of three polymorphic isotypes of the class II major histocompatibility complex (MHC) antigen. Because HLA-DR is expressed at high levels on a range of hematologic malignancies, there has been considerable interest in its development as a target for antibody-based lymphoma therapy. However, safety concerns have been raised regarding the clinical use of HLA-DR-directed antibodies, because the antigen is expressed on normal as well as tumor cells. (Dechant et al., 2003, Semin Oncol 30:465-75) HLA-DR is constitutively expressed on normal B cells, monocytes/macrophages, dendritic cells, and thymic epithelial cells. In addition, interferon-gamma may induce HLA class II expression on other cell types, including activated T and endothelial cells (Dechant et al., 2003). The most widely recognized function of HLA molecules is the presentation of antigen in the form of short peptides to the antigen receptor of T lymphocytes. In addition, signals delivered via HLA-DR molecules contribute to the functioning of the immune system by up-regulating the activity of adhesion molecules, inducing T-cell antigen counterreceptors, and initiating the synthesis of cytokines. (Nagy and Mooney, 2003, J Mol Med 81:757-65; Scholl et al., 1994, Immunol Today 15:418-22)

The CD74 antigen is an epitope of the major histocompatibility complex (MHC) class II antigen invariant chain, Ii, present on the cell surface and taken up in large amounts of up to 8×10⁶ molecules per cell per day (Hansen et al., 1996, Biochem. J., 320: 293-300). CD74 is present on the cell surface of B-lymphocytes, monocytes and histocytes, human B-lymphoma cell lines, melanomas, T-cell lymphomas and a variety of other tumor cell types. (Hansen et al., 1996, Biochem. J., 320: 293-300) CD74 associates with α/β chain MHC II heterodimers to form MHC II αβIi complexes that are involved in antigen processing and presentation to T cells (Dixon et al., 2006, Biochemistry 45:5228-34; Loss et al., 1993, J Immunol 150:3187-97; Cresswell et al., 1996; Cell 84:505-7).

CD74 plays an important role in cell proliferation and survival. Binding of the CD74 ligand, macrophage migration inhibitory factor (MIF), to CD74 activates the MAP kinase cascade and promotes cell proliferation (Leng et al., 2003, J Exp Med 197:1467-76). Binding of MIF to CD74 also enhances cell survival through activation of NF-κB and Bcl-2 (Lantner et al., 2007, Blood 110:4303-11).

Antibodies against CD74 and/or HLA-DR have been reported to show efficacy against cancer cells. Such anti-cancer antibodies include the anti-CD74 hLL1 antibody (milatuzumab) and the anti-HLA-DR antibody hL243 (also known as IMMU-114) (Berkova et al., Expert Opin. Investig. Drugs 19:141-49; Burton et al., 2004, Clin Cancer Res 10:6605-11; Chang et al., 2005, Blood 106:4308-14; Griffiths et al., 2003, Clin Cancer Res 9:6567-71; Stein et al., 2007, Clin Cancer Res 13:5556s-63s; Stein et al., 2010, Blood 115:5180-90). However, despite the efficacy of such antibodies against cancer, some types of CD74 and/or HLA-DR expressing cancers have been reported to be resistant to antibody therapy (see, e.g., Stein et al., 2010, Blood 115:5180-90). A need exists for more effective methods and compositions for therapeutic use of anti-CD74 and/or anti-HLA-DR antibodies.

### SUMMARY

The present invention concerns improved compositions and methods of use of anti-CD74 antibodies or antigen-binding antibody fragments. In preferred embodiments, the compositions and methods include interferon-gamma, which may be administered prior to or concurrently with the anti-CD74 antibodies or fragments thereof. More preferably, the administration of interferon-gamma increases the expression of CD74 and enhances the sensitivity of cancer cells, autoimmune disease cells or immune dysfunction cells to the cytotoxic effects of anti-CD74 antibodies.

Many examples of anti-CD74 antibodies are known in the art and any such known antibody or fragment thereof may be utilized. In a preferred embodiment, the anti-CD74 antibody is an hLL1 antibody (also known as milatuzumab) that comprises the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 4. However, in alternative embodiments, other known anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN.1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other anti-CD74 antibody known in the art.

The anti-CD74 antibody may be selected such that it competes with or blocks binding to CD74 of an LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). Alternatively, the anti-CD74 antibody may bind to the same epitope of CD74 as an LL1 antibody. In still other alternatives, the anti-CD74 antibody may exhibit a functional characteristic such as internalization by Raji lymphoma cells in culture or inducing apoptosis of Raji cells in cell culture when cross-linked.

Alternative embodiments may involve use of anti-HLA-DR antibodies or fragments thereof and treatment with interferon-gamma to increase expression of HLA-DR and enhance sensitivity of cancer or autoimmune disease cells to anti-HLA-DR antibodies. Many examples of anti-HLA-DR antibodies are known in the art and any such known antibody or fragment thereof may be utilized. In a preferred embodiment, the anti-HLA-DR antibody is an hL243 antibody (also known as IMMU-114) that comprises the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). A humanized L243 anti-HLA-DR antibody suitable for use is disclosed in U.S. Pat. No. 7,612,180, incorporated herein by reference from Col. 46, line 45 through Col. 60, line 50 and FIG. 1 through FIG. 6. However, in alternative embodiments, other known anti-HLA-DR antibodies may be utilized, such as 1D10 (apolizumab) (Kostelny et al., 2001, Int J Cancer 93:556-65); MS-GPC-1, MS-GPC-6, MS-GPC-8, MS-GPC-10, etc. (U.S. Pat. No. 7,521,047); Lym-1, TAL 8.1, 520B, ML11C11, SPM289, MEM-267, TAL 15.1, TAL 1B5, G-7, 4D12, Bra30, etc. (Santa Cruz Biotechnology, Inc., Santa Cruz, Calif.); TAL 16.1, TU36, C120 (ABCAM®, Cambridge, Mass.); and any other anti-HLA-DR antibody known in the art.

The anti-HLA-DR antibody may be selected such that it competes with or blocks binding to HLA-DR of an L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). Alternatively, the anti-HLA-DR antibody may bind to the same epitope of HLA-DR as an L243 antibody.

The anti-CD74 and/or anti-HLA-DR antibodies or fragments thereof may be used as naked antibodies, alone or in combination with one or more therapeutic agents. Alternatively, the antibodies or fragments may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene) or a second antibody or fragment thereof.

Antisense molecules may include antisense molecules that correspond to bcl-2 or p53. However, other antisense molecules are known in the art, as described below, and any such known antisense molecule may be used. Second antibodies or fragments thereof may bind to an antigen selected from the group consisting of carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CXCR4, CXCR7, CXCL12, HIF-1α, AFP, PSMA, CEACAM5, CEACAM6, B7, ED-B of fibronectin, Factor H, FHL-1, Flt-3, folate receptor, GROB, HMGB-1, hypoxia inducible factor (HIF), HM1.24, insulin-like growth factor-1 (IGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, MAGE, mCRP, MCP-1, MIP-1A, MIP-1B, MIF, MUC1, MUC2, MUC3, MUC4, MUC5, NCA-95, NCA-90, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, P1GF, complement factors C3, C3a, C3b, C5a, C5, and an oncogene product.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

The therapeutic agent may comprise a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a stem cell growth factor, a lymphotoxin, a hematopoietic factor, a colony stimulating factor (CSF), an interferon (IFN), erythropoietin, thrombopoietin and combinations thereof. Exemplary immunomodulators may include IL-1, IL-2, IL-3, IL-6, IL-10, IL-12, IL-18, IL-21, interferon-α, interferon-β, interferon-γ, G-CSF, GM-CSF, and mixtures thereof.

Exemplary anti-angiogenic agents may include angiostatin, endostatin, basculostatin, canstatin, maspin, anti-VEGF binding molecules, anti-placental growth factor binding molecules, or anti-vascular growth factor binding molecules.

In certain embodiments, the antibody or fragment may comprise one or more chelating moieties, such as NOTA, DOTA, DTPA, TETA, Tscg-Cys, or Tsca-Cys. In certain embodiments, the chelating moiety may form a complex with a therapeutic or diagnostic cation, such as Group II, Group III, Group IV, Group V, transition, lanthanide or actinide metal cations, Tc, Re, Bi, Cu, As, Ag, Au, At, or Pb.

In some embodiments, the antibody or fragment thereof may be a human, chimeric, or humanized antibody or fragment thereof. A humanized antibody or fragment thereof may comprise the complementarity-determining regions (CDRs) of a murine antibody and the constant and framework (FR) region sequences of a human antibody, which may be substituted with at least one amino acid from corresponding FRs of a murine antibody. A chimeric antibody or fragment thereof may include the light and heavy chain variable regions of a murine antibody, attached to human antibody constant regions. The antibody or fragment thereof may include human constant regions of IgG1, IgG2a, IgG3, or IgG4.

In certain preferred embodiments, the anti-CD74 or anti-HLA-DR complex may be formed by a technique known as dock-and-lock (DNL) (see, e.g., U.S. Pat. Nos. 7,521,056; 7,527,787; 7,534,866; 7,550,143 and U.S. Patent Publ. No. 20090060862, filed Oct. 26, 2007, the Examples section of each of which is incorporated herein by reference.) Generally, the DNL technique takes advantage of the specific and high-affinity binding interaction between a dimerization and docking domain (DDD) sequence derived from cAMP-dependent protein kinase and an anchor domain (AD) sequence derived from any of a variety of AKAP proteins. The DDD and AD peptides may be attached to any protein, peptide or other molecule. Because the DDD sequences spontaneously dimerize and bind to the AD sequence, the DNL technique allows the formation of complexes between any selected molecules that may be attached to DDD or AD sequences. Although the standard DNL complex comprises a trimer with two DDD-linked molecules attached to one AD-linked molecule, variations in complex structure allow the formation of dimers, trimers, tetramers, pentamers, hexamers and other multimers. In some embodiments, the DNL complex may comprise two or more antibodies, antibody fragments or fusion proteins which bind to the same antigenic determinant or to two or more different antigens. The DNL complex may also comprise one or more other effectors, such as a cytokine or PEG moiety.

Also disclosed is a method for treating and/or diagnosing a disease or disorder that includes administering to a patient a therapeutic and/or diagnostic composition that includes any of the aforementioned antibodies or fragments thereof. Typically, the composition is administered to the patient intravenously, intramuscularly or subcutaneously at a dose of 20-5000 mg.

In preferred embodiments, the disease or disorder is associated with CD74- and/or HLA-DR-expressing cells and may be a cancer, an immune dysregulation disease, an autoimmune disease, an organ-graft rejection, a graft-versus-host disease, a solid tumor, non-Hodgkin's lymphoma, Hodgkin's lymphoma, multiple myeloma, a B-cell malignancy, or a T-cell malignancy. A B-cell malignancy may-include indolent forms of B-cell lymphomas, aggressive forms of B-cell lymphomas, chronic lymphatic leukemias, acute lymphatic leukemias, and/or multiple myeloma. Solid tumors may include melanomas, carcinomas, sarcomas, and/or gliomas. A carcinoma may include renal carcinoma, lung carcinoma, intestinal carcinoma, stomach carcinoma, breast carcinoma, prostate cancer, ovarian cancer, and/or melanoma.

Exemplary autoimmune diseases include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis. However, the skilled artisan will realize that any disease or condition characterized by expression of CD74 and/or HLA-DR may be treated using the claimed compositions and methods.

### BRIEF DESCRIPTION OF THE DRAWINGS

FIG. 1. Immunostaining for CD74 expression in tissue samples from AML cases. Trephine bone marrow biopsy slides were deparaffinized with xylene and sequentially re-hydrated. They were then treated with 0.1% hydrogen peroxide to block endogenous peroxidase and were then blocked with BSA/FCS buffer and reacted with optimal dilutions of LL1 and control MAb. After washing, pre-titered 2nd antibody (goat anti-mouse peroxidase) was added. After washing, DAB reagent was added for color development.

FIG. 2. Upregulation of CD74 by IFN-γ assayed by flow cytometry. (A) GDM-1 and (B) Kasumi-1 AML cell lines were cultured under standard conditions or with IFN-γ, harvested and stained with control or hLL1 MAb by an indirect method (comparisons with no IFN-γ-GDM-1: P=0.0003; Kasumi-1: P<0.001; MCF=Mean Fluorescence channel).

FIG. 3. Anti-proliferative effect of milatuzumab in (A) GDM-1 and (B) Kasumi-1 AML cell lines with or without IFN-γ, as determined by MTT assay. AML lines were added to 96-well plates, to which media, with or without IFN-γ, hLL 1 and crosslinking antibody (goat anti-human) was added. Plates were incubated for 5 days, when MTT was added followed by determination of OD values. Student t-test comparisons of no IFN-γ with 40 and 200 U/mL (GDM-1-P=0.01; Kasumi-1: P<0.05).

FIG. 4. Apoptotic effect of milatuzumab in (A) GDM-1 and (B) Kasumi-1 AML cell lines with or without IFN-γ, as determined by annexin V assay. AML lines were cultured in media with or without IFN-γ, hLL1 and crosslinking (goat anti-human) antibody for 2 days, and then were stained with FITC-labeled Annexin V and analyzed by flow cytometry. Since growth inhibitory effects were increased with IFN-γ and crosslinking antibody, these data are presented. P values for comparisons with both cell lines were <0.05.

FIG. 5. Therapy with different antibodies in NHL-bearing SCID mice. Protocol: 250 mg of the indicated MAb/injection, 2×/wk for 4 wks, starting 1 day after injection of WSU-FSCCL tumor cells. During our previous work on anti-B-cell MAbs, we observed that the anti-HLA-DR and anti-CD74 MAbs, hL243g4P and milatuzumab, had potent therapeutic activity toward B-cell malignancies. In the representative data shown here, SCID mice bearing WSU-FSCCL follicular lymphoma are more sensitive to these two MAbs than to anti-CD20 MAbs such as rituximab.

FIG. 6. Cytotoxicity comparisons with anti-CD74 and anti-HLA-DR antibodies in the presence or absence of IFN-γ.

FIG. 7. Ex vivo effects of MAbs on whole blood. Heparinized whole blood of healthy volunteers was incubated with MAbs then assayed by flow cytometry. Data are shown as % of untreated control. Error bars, SD of 3 replicates. *, P<0.05 relative to no MAb control.

FIG. 8. Effect of ERK, JNK and ROS inhibitors on hL234g4P mediated apoptosis in Raji cells.

### DETAILED DESCRIPTION

### Definitions

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody) or an immunologically active (i.e., antigen-binding) portion of an immunoglobulin molecule, like an antibody fragment.

An “antibody fragment” is a portion of an antibody such as F(ab′)₂, F(ab)₂, Fab′, Fab, Fv, scFv, single domain antibodies (DABS or VHHs) and the like, including half-molecules of IgG4 (van der Neut Kolfschoten et al. (Science 2007; 317(14 September):1554-1557). Regardless of structure, an antibody fragment binds with the same antigen that is recognized by the intact antibody. For example, an anti-CD74 antibody fragment binds with an epitope of CD74. The term “antibody fragment” also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains. Additional FR amino acid substitutions from the parent, e.g. murine, antibody may be made. The constant domains of the antibody molecule are derived from those of a human antibody.

A “human antibody” is an antibody obtained from transgenic mice that have been genetically engineered to produce human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, toxins, enzymes, nucleases, hormones, immunomodulators, antisense oligonucleotides, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody, antibody fragment, antibody fusion protein, bispecific antibody or multispecific antibody with an atom, molecule, or a higher-ordered structure (e.g., with a carrier, a therapeutic agent, or a diagnostic agent). A “naked antibody” is an antibody that is not conjugated to any other agent.

As used herein, the term “antibody fusion protein” is a recombinantly produced antigen-binding molecule in which an antibody or antibody fragment is linked to another protein or peptide, such as the same or different antibody or antibody fragment or a DDD or AD peptide. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity. For example, a diabody, where one binding site reacts with one antigen and the other with another antigen.

A “bispecific antibody” is an antibody that can bind simultaneously to two targets which are of different structure. Bispecific antibodies (bsAb) and bispecific antibody fragments (bsFab) may have at least one arm that specifically binds to, for example, a B-cell, T-cell, myeloid-, plasma-, and mast-cell antigen or epitope and at least one other arm that specifically binds to a targetable conjugate that bears a therapeutic or diagnostic agent. A variety of bispecific antibodies can be produced using molecular engineering.

Preparation of Antibodies

The immunoconjugates and compositions described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)).

General techniques for cloning murine immunoglobulin variable domains have been disclosed, for example, by the publication of Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG₁ constant region domains. This publication also provides the nucleotide sequences of the LL2 light and heavy chain variable regions, Vₖ and V_{H}, respectively. Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

A chimeric antibody is a recombinant protein that contains the variable domains including the CDRs derived from one species of animal, such as a rodent antibody, while the remainder of the antibody molecule; i.e., the constant domains, is derived from a human antibody. Accordingly, a chimeric monoclonal antibody can also be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)).

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al, (2005) constructed a phage display library of human Fab antibody fragments from osteosarcoma patients. Generally, total RNA was obtained from circulating blood lymphocytes (Id.) Recombinant Fab were cloned from the μ, γ and κ chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J Immunol. Methods 231:11-23, incorporated herein by reference) from Abgenix (Fremont, Calif.). In the XENOMOUSE® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

Known Antibodies

In various embodiments, the claimed methods and compositions may utilize any of a variety of antibodies known in the art. Antibodies of use may be commercially obtained from a number of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,15; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206′ 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art.

Particular antibodies that may be of use for therapy of cancer within the scope of the claimed methods and compositions include, but are not limited to, LL1 (anti-CD74), LL2 and RFB4 (anti-CD22), RS7 (anti-epithelial glycoprotein-1 (EGP-1)), PAM4 and KC4 (both anti-mucin), MN-14 (anti-carcinoembryonic antigen (CEA, also known as CD66e)), Mu-9 (anti-colon-specific antigen-p), Immu-31 (an anti-alpha-fetoprotein), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb) and hL243 (anti-HLA-DR). Such antibodies are known in the art (e.g., U.S. Pat. Nos. 5,686,072; 5,874,540; 6,107,090; 6,183,744; 6,306,393; 6,653,104; 6,730,300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20040202666 (now abandoned); 20050271671; and 20060193865; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU31 (U.S. Pat. No. 7,300,655), hLL1 (U.S. Pat. No. 7,312,318,), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. Provisional Patent Application 61/145,896), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797).

Multispecific and Multivalent Antibodies

Various embodiments may concern use of multispecific and/or multivalent antibodies. For example, an anti-CD74 antibody or fragment thereof and an anti-HLA-DR antibody or fragment thereof may be joined together by means such as the dock-and-lock technique described below. Other combinations of antibodies or fragments thereof may be utilized. For example, another antigen expressed by the CD74- or HLA-DR-expressing cell may include a tumor marker selected from a B-cell lineage antigen, (e.g., CD19, CD20, or CD22 for the treatment of B-cell malignancies). The tumor cell marker may be a non-B-cell lineage antigen selected from the group consisting of HLA-DR, CD30, CD33, CD52 MUC1, MUC5 and TAC. Other useful antigens may include carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CXCR4, CXCR7, CXCL12, HIF-1α, CEACAM5, CEACAM-6, alpha-fetoprotein (AFP), VEGF (e.g. AVASTIN®, fibronectin splice variant), ED-B fibronectin (e.g., L19), EGP-1, EGP-2 (e.g., 17-1A), EGF receptor (ErbB1) (e.g., ERBITUX®), ErbB2, ErbB3, Factor H, FHL-1, Flt-3, folate receptor, Ga 733, GROB, HMGB-1, hypoxia inducible factor (HIF), HM1.24, HER-2/neu, insulin-like growth factor (IGF), IFN-γ, IFN-α, IFN-β, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, pancreatic cancer mucin, pancreatic cancer mucin, NCA-95, NCA-90, A3, A33, Ep-CAM, KS-1, Le(y), mesothelin, 5100, tenascin, TAC, Tn antigen, Thomas-Friedenreich antigens, tumor necrosis antigens, tumor angiogenesis antigens, TNF-α, TRAIL receptor (R1 and R2), VEGFR, RANTES, T101, as well as cancer stem cell antigens, complement factors C3, C3a, C3b, C5a, C5, and an oncogene product.

Methods for producing bispecific antibodies include engineered recombinant antibodies which have additional cysteine residues so that they crosslink more strongly than the more common immunoglobulin isotypes. (See, e.g., FitzGerald et al, Protein Eng. 10(10):1221-1225, (1997)). Another approach is to engineer recombinant fusion proteins linking two or more different single-chain antibody or antibody fragment segments with the needed dual specificities. (See, e.g., Coloma et al., Nature Biotech. 15:159-163, (1997)). A variety of bispecific antibodies can be produced using molecular engineering. In one form, the bispecific antibody may consist of, for example, an scFv with a single binding site for one antigen and a Fab fragment with a single binding site for a second antigen. In another form, the bispecific antibody may consist of, for example, an IgG with two binding sites for one antigen and two scFv with two binding sites for a second antigen.

Diabodies, Triabodies and Tetrabodies

The compositions disclosed herein may also include functional bispecific single-chain antibodies (bscAb), also called diabodies. (See, e.g., Mack et al., Proc. Natl. Acad. Sci., 92.: 7021-7025, 1995). For example, bscAb are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNAs obtained from each hybridoma are then joined to form a single-chain fragment in a two-step fusion PCR. The first PCR step introduces the (Gly₄-Ser₁)₃ linker (SEQ ID NO: 96), and the second step joins the V_{L} and V_{H} amplicons. Each single chain molecule is then cloned into a bacterial expression vector. Following amplification, one of the single-chain molecules is excised and sub-cloned into the other vector, containing the second single-chain molecule of interest. The resulting bscAb fragment is subcloned into a eukaryotic expression vector. Functional protein expression can be obtained by transfecting the vector into Chinese Hamster Ovary cells.

For example, a humanized, chimeric or human anti-CD74 monoclonal antibody can be used to produce antigen specific diabodies, triabodies, and tetrabodies. The monospecific diabodies, triabodies, and tetrabodies bind selectively to targeted antigens and as the number of binding sites on the molecule increases, the affinity for the target cell increases and a longer residence time is observed at the desired location. For diabodies, the two chains comprising the V_{H} polypeptide of the humanized CD74 antibody connected to the V_{K} polypeptide of the humanized CD74 antibody by a five amino acid residue linker may be utilized. Each chain forms one half of the humanized CD74 diabody. In the case of triabodies, the three chains comprising V_{H} polypeptide of the humanized CD74 antibody connected to the V_{K} polypeptide of the humanized CD74 antibody by no linker may be utilized. Each chain forms one third of the hCD74 triabody.

More recently, a tetravalent tandem diabody (termed tandab) with dual specificity has also been reported (Cochlovius et al., Cancer Research (2000) 60: 4336-4341). The bispecific tandab is a dimer of two identical polypeptides, each containing four variable domains of two different antibodies (V_{H1}, V_{L1}, V_{H2}, V_{L2}) linked in an orientation to facilitate the formation of two potential binding sites for each of the two different specificities upon self-association.

### Dock-and-Lock (DNL)

In certain preferred embodiments, bispecific or multispecific antibodies may be produced using the dock-and-lock (DNL) technology (see, e.g., U.S. Pat. Nos. 7,521,056; 7,550,143; 7,534,866; 7,527,787 and 7,666,400; the Examples section of each of which is incorporated herein by reference). The DNL method exploits specific protein/protein interactions that occur between the regulatory (R) subunits of cAMP-dependent protein kinase (PKA) and the anchoring domain (AD) of A-kinase anchoring proteins (AKAPs) (Baillie et al., FEBS Letters. 2005; 579: 3264. Wong and Scott, Nat. Rev. Mol. Cell. Biol. 2004; 5: 959). PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). The R subunits have been isolated only as stable dimers and the dimerization domain has been shown to consist of the first 44 amino-terminal residues (Newlon et al., Nat. Struct. Biol. 1999; 6:222). Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561)

Since the first AKAP, microtubule-associated protein-2, was characterized in 1984 (Lohmann et al., Proc. Natl. Acad. Sci. USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell. Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIa are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human RIIα and the AD of AKAP as an excellent pair of linker modules for docking any two entities, referred to hereafter as A and B, into a noncovalent complex, which could be further locked into a stably tethered structure through the introduction of cysteine residues into both the DDD and AD at strategic positions to facilitate the formation of disulfide bonds. The general methodology of the “dock-and-lock” approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL constructs of different stoichiometry may be produced and used, including but not limited to dimeric, trimeric, tetrameric, pentameric and hexameric DNL constructs (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

Pre-Targeting

In certain alternative embodiments, therapeutic agents may be administered by a pretargeting method, utilizing bispecific or multispecific antibodies. In pretargeting, the bispecific or multispecific antibody comprises at least one binding arm that binds to an antigen exhibited by a targeted cell or tissue, while at least one other binding arm binds to a hapten on a targetable construct. The targetable construct comprises one or more haptens and one or more therapeutic and/or diagnostic agents.

Pre-targeting is a multistep process originally developed to resolve the slow blood clearance of directly targeting antibodies, which contributes to undesirable toxicity to normal tissues such as bone marrow. With pre-targeting, a radionuclide or other diagnostic or therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Immunoconjugates

In preferred embodiments, an antibody or antibody fragment may be directly attached to one or more therapeutic agents to form an immunoconjugate. Therapeutic agents may be attached, for example to reduced SH groups and/or to carbohydrate side chains. A therapeutic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic agent can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching therapeutic agents to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted akyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.) These and other known click chemistry reactions may be used to attach therapeutic agents to antibodies in vitro.

The specificity of the click chemistry reaction may be used as a substitute for the antibody-hapten binding interaction used in pretargeting with bispecific antibodies. In this alternative embodiment, the specific reactivity of e.g., cyclooctyne moieties for azide moieties or alkyne moieties for nitrone moieties may be used in an in vivo cycloaddition reaction. An antibody or other targeting molecule is activated by incorporation of a substituted cyclooctyne, an azide or a nitrone moiety. A targetable construct is labeled with one or more diagnostic or therapeutic agents and a complementary reactive moiety. I.e., where the targeting molecule comprises a cyclooctyne, the targetable construct will comprise an azide; where the targeting molecule comprises a nitrone, the targetable construct will comprise an alkyne, etc. The activated targeting molecule is administered to a subject and allowed to localize to a targeted cell, tissue or pathogen, as disclosed for pretargeting protocols. The reactive labeled targetable construct is then administered. Because the cyclooctyne, nitrone or azide on the targetable construct is unreactive with endogenous biomolecules and highly reactive with the complementary moiety on the targeting molecule, the specificity of the binding interaction results in the highly specific binding of the targetable construct to the tissue-localized targeting molecule.

Therapeutic Agents

A wide variety of therapeutic reagents can be administered concurrently or sequentially with the subject anti-CD74 and/or anti-HLA-DR antibodies. For example, drugs, toxins, oligonucleotides, immunomodulators, hormones, hormone antagonists, enzymes, enzyme inhibitors, radionuclides, angiogenesis inhibitors, other antibodies or fragments thereof, etc. The therapeutic agents recited here are those agents that also are useful for administration separately with an antibody or fragment thereof as described above. Therapeutic agents include, for example, chemotherapeutic drugs such as vinca alkaloids, anthracyclines, gemcitabine, epipodophyllotoxins, taxanes, antimetabolites, alkylating agents, antibiotics, SN-38, COX-2 inhibitors, antimitotics, anti-angiogenic and pro-apoptotic agents, particularly doxorubicin, methotrexate, taxol, CPT-11, camptothecans, proteosome inhibitors, mTOR inhibitors, HDAC inhibitors, tyrosine kinase inhibitors, and others.

Other useful cancer chemotherapeutic drugs include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HDAC inhibitors, camptothecins, hormones, and the like. Suitable chemotherapeutic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications. Other suitable chemotherapeutic agents, such as experimental drugs, are known to those of skill in the art.

In a preferred embodiment, conjugates of camptothecins and related compounds, such as SN-38, may be conjugated to an anti-cancer antibody, for example as disclosed in U.S. Pat. No. 7,591,994; and U.S. Ser. No. 11/388,032, filed Mar. 23, 2006, the Examples section of each of which is incorporated herein by reference.

A toxin can be of animal, plant or microbial origin. A toxin, such as Pseudomonas exotoxin, may also be complexed to or form the therapeutic agent portion of an immunoconjugate. Other toxins include ricin, abrin, ribonuclease (RNase), DNase I, Staphylococcal enterotoxin-A, pokeweed antiviral protein, onconase, gelonin, diphtheria toxin, Pseudomonas exotoxin, and Pseudomonas endotoxin. See, for example, Pastan et al., Cell 47:641 (1986), Goldenberg, CA—A Cancer Journal for Clinicians 44:43 (1994), Sharkey and Goldenberg, CA—A Cancer Journal for Clinicians 56:226 (2006). Additional toxins suitable for use are known to those of skill in the art and are disclosed in U.S. Pat. No. 6,077,499, the Examples section of which is incorporated herein by reference.

As used herein, the term “immunomodulator” includes a cytokine, a lymphokine, a monokine, a stem cell growth factor, a lymphotoxin, a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), TGF-α, TGF-β, insulin-like growth factor (IGF), erythropoietin, thrombopoietin, tumor necrosis factor (TNF), TNF-α, TNF-β, a mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, S1 factor, IL-1, IL-1 cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 and IL-25, LIF, kit-ligand, FLT-3, angiostatin, thrombospondin, endostatin and LT, and the like.

The antibody or fragment thereof may be administered as an immunoconjugate comprising one or more radioactive isotopes useful for treating diseased tissue. Particularly useful therapeutic radionuclides include, but are not limited to ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, I-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV.

Additional potential therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

Interference RNA

In certain preferred embodiments the therapeutic agent may be a siRNA or interference RNA species. The siRNA, interference RNA or therapeutic gene may be attached to a carrier moiety that is conjugated to an antibody or fragment thereof. A variety of carrier moieties for siRNA have been reported and any such known carrier may be incorporated into a therapeutic antibody for use. Non-limiting examples of carriers include protamine (Rossi, 2005, Nat Biotech 23:682-84; Song et al., 2005, Nat Biotech 23:709-17); dendrimers such as PAMAM dendrimers (Pan et al., 2007, Cancer Res. 67:8156-8163); polyethylenimine (Schiffelers et al., 2004, Nucl Acids Res 32:e149); polypropyleneimine (Taratula et al., 2009, J Control Release 140:284-93); polylysine (Inoue et al., 2008, J Control Release 126:59-66); histidine-containing reducible polycations (Stevenson et al., 2008, J Control Release 130:46-56); histone H1 protein (Haberland et al., 2009, Mol Biol Rep 26:1083-93); cationic comb-type copolymers (Sato et al., 2007, J Control Release 122:209-16); polymeric micelles (U.S. Patent Application Publ. No. 20100121043); and chitosan-thiamine pyrophosphate (Rojanarata et al., 2008, Pharm Res 25:2807-14). The skilled artisan will realize that in general, polycationic proteins or polymers are of use as siRNA carriers. The skilled artisan will further realize that siRNA carriers can also be used to carry other oligonucleotide or nucleic acid species, such as anti-sense oligonucleotides or short DNA genes.

Known siRNA species of potential use include those specific for IKK-gamma (U.S. Pat. No. 7,022,828); VEGF, Flt-1 and Flk-1/KDR (U.S. Pat. No. 7,148,342); Bc12 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL complexes.

Exemplary siRNA species known in the art are listed in Table 1. Although siRNA is delivered as a double-stranded molecule, for simplicity only the sense strand sequences are shown in Table 1.

TABLE 1
Exemplary siRNA Sequences

| Target | Sequence | SEQ ID NO |
|---|---|---|
| VEGF R2 | AATGCGGCGGTGGTGACAGTA | SEQ ID NO: 13 |
| VEGF R2 | AAGCTCAGCACACAGAAAGAC | SEQ ID NO: 14 |
| CXCR4 | UAAAAUCUUCCUGCCCACCdTdT | SEQ ID NO: 15 |
| CXCR4 | GGAAGCUGUUGGCUGAAAAdTdT | SEQ ID NO: 16 |
| PPARC1 | AAGACCAGCCUCUUUGCCCAG | SEQ ID NO: 17 |
| Dynamin 2 | GGACCAGGCAGAAAACGAG | SEQ ID NO: 18 |
| Catenin | CUAUCAGGAUGACGCGG | SEQ ID NO: 19 |
| E1A binding protein | UGACACAGGCAGGCUUGACUU | SEQ ID NO: 20 |
| Plasminogen | GGTGAAGAAGGGCGTCCAA | SEQ ID NO: 21 |
| activator | | |
| K-ras | GATCCGTTGGAGCTGTTGGCGTAGTT | SEQ ID NO: 22 |
| | CAAGAGACTCGCCAACAGCTCCAACT | |
| | TTTGGAAA | |
| Sortilin 1 | AGGTGGTGTTAACAGCAGAG | SEQ ID NO: 23 |
| Apolipoprotein E | AAGGTGGAGCAAGCGGTGGAG | SEQ ID NO: 24 |
| Apolipoprotein E | AAGGAGTTGAAGGCCGACAAA | SEQ ID NO: 25 |
| Bcl-X | UAUGGAGCUGCAGAGGAUGdTdT | SEQ ID NO: 26 |
| Raf-1 | TTTGAATATCTGTGCTGAGAACACA | SEQ ID NO: 27 |
| | GTTCTCAGCACAGATATTCTTTTT | |
| Heat shock | AATGAGAAAAGCAAAAGGTGCCCTGTCTC | SEQ ID NO: 28 |
| transcription factor 2 | | |
| IGFBP3 | AAUCAUCAUCAAGAAAGGGCA | SEQ ID NO: 29 |
| Thioredoxin | AUGACUGUCAGGAUGUUGCdTdT | SEQ ID NO: 30 |
| CD44 | GAACGAAUCCUGAAGACAUCU | SEQ ID NO: 31 |
| MMP14 | AAGCCTGGCTACAGCAATATGCCTGTCTC | SEQ ID NO: 32 |
| MAPKAPK2 | UGACCAUCACCGAGUUUAUdTdT | SEQ ID NO: 33 |
| FGFR1 | AAGTCGGACGCAACAGAGAAA | SEQ ID NO: 34 |
| ERBB2 | CUACCUUUCUACGGACGUGdTdT | SEQ ID NO: 35 |
| BCL2L1 | CTGCCTAAGGCGGATTTGAAT | SEQ ID NO: 36 |
| ABL1 | TTAUUCCUUCUUCGGGAAGUC | SEQ ID NO: 37 |
| CEACAM1 | AACCTTCTGGAACCCGCCCAC | SEQ ID NO: 38 |
| CD9 | GAGCATCTTCGAGCAAGAA | SEQ ID NO: 39 |
| CD151 | CATGTGGCACCGTTTGCCT | SEQ ID NO: 40 |
| Caspase 8 | AACTACCAGAAAGGTATACCT | SEQ ID NO: 41 |
| BRCA1 | UCACAGUGUCCUUUAUGUAdTdT | SEQ ID NO: 42 |
| p53 | GCAUGAACCGGAGGCCCAUTT | SEQ ID NO: 43 |
| CEACAM6 | CCGGACAGTTCCATGTATA | SEQ ID NO: 44 |

The skilled artisan will realize that Table 1 represents a very small sampling of the total number of siRNA species known in the art, and that any such known siRNA may be utilized in the claimed methods and compositions.

Methods of Therapeutic Treatment

The methods and compositions are of use for treating disease states, such as cancer, autoimmune disease or immune dysfunction. The methods may comprise administering a therapeutically effective amount of a therapeutic antibody or fragment thereof or an immunoconjugate, either alone or in conjunction with one or more other therapeutic agents, administered either concurrently or sequentially.

Multimodal therapies may include therapy with other antibodies, such as anti-CD22, anti-CD19, anti-CD20, anti-CD21, anti-CD74, anti-CD80, anti-CD23, anti-CD45, anti-CD46, anti-MIF, anti-EGP-1, anti-CEACAM5, anti-CEACAM6, anti-pancreatic cancer mucin, anti-IGF-1R or anti-HLA-DR (including the invariant chain) antibodies in the form of naked antibodies, fusion proteins, or as immunoconjugates. Various antibodies of use, such as anti-CD19, anti-CD20, and anti-CD22 antibodies, are known to those of skill in the art. See, for example, Ghetie et al., Cancer Res. 48:2610 (1988); Hekman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,612,180; 7,501,498; the Examples section of each of which is incorporated herein by reference.

In another form of multimodal therapy, subjects receive therapeutic antibodies in conjunction with standard cancer chemotherapy. For example, “CVB” (1.5 g/m² cyclophosphamide, 200-400 mg/m² etoposide, and 150-200 mg/m² carmustine) is a regimen used to treat non-Hodgkin's lymphoma. Patti et al., Eur. I Haematol. 51: 18 (1993). Other suitable combination chemotherapeutic regimens are well-known to those of skill in the art. See, for example, Freedman et al., “Non-Hodgkin's Lymphomas,” in CANCER MEDICINE, VOLUME 2, 3rd Edition, Holland et al. (eds.), pages 2028-2068 (Lea & Febiger 1993). As an illustration, first generation chemotherapeutic regimens for treatment of intermediate-grade non-Hodgkin's lymphoma (NHL) include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate, bendamustine, and bryostatin-1.

In a preferred multimodal therapy, both chemotherapeutic drugs and cytokines are co-administered with a therapeutic antibody. The cytokines, chemotherapeutic drugs and therapeutic antibody can be administered in any order, or together.

Therapeutic antibodies or fragments thereof can be formulated according to known methods to prepare pharmaceutically useful compositions, whereby the therapeutic antibody is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The therapeutic antibody can be formulated for intravenous administration via, for example, bolus injection or continuous infusion. Preferably, the therapeutic antibody is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The therapeutic antibody may also be administered to a mammal subcutaneously or even by other parenteral routes. Moreover, the administration may be by continuous infusion or by single or multiple boluses. Preferably, the therapeutic antibody is infused over a period of less than about 4 hours, and more preferably, over a period of less than about 3 hours.

More generally, the dosage of an administered therapeutic antibody for humans will vary depending upon such factors as the patient's age, weight, height, sex, general medical condition and previous medical history. It may be desirable to provide the recipient with a dosage of therapeutic antibody that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, a therapeutic antibody may be administered as one dosage every 2 or 3 weeks, repeated for a total of at least 3 dosages. Or, the therapeutic antibody may be administered twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

Additional pharmaceutical methods may be employed to control the duration of action of the therapeutic immunoconjugate or naked antibody. Control release preparations can be prepared through the use of polymers to complex or adsorb the immunoconjugate or naked antibody. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release of an immunoconjugate or antibody from such a matrix depends upon the molecular weight of the immunoconjugate or antibody, the amount of immunoconjugate or antibody within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Cancer Therapy

In preferred embodiments, the antibodies, antibody fragments or immunoconjugates are of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Gerin Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

Therapy of Autoimmune Disease

Anti-CD74 and/or anti-HLA-DR antibodies or immunoconjugates can be used to treat immune dysregulation disease and related autoimmune diseases, including Class-III autoimmune diseases, immune-mediated thrombocytopenias, such as acute idiopathic thrombocytopenic purpura and chronic idiopathic thrombocytopenic purpura, dermatomyositis, Sjogren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis obliterans, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

### EXAMPLES

Various embodiments of the present invention are illustrated by the following examples, without limiting the scope thereof.

### Example 1

### Expression of CD74 by AML Blasts and Cell Lines and Enhanced Cytotoxicity of Anti-CD74 Antibodies After Interferon-Gamma (IFN-γ) Treatment

CD74 (invariant chain, Ii) is a type-II transmembrane glycoprotein that associates with the major histocompatibility class (MHC) II α and β chains and directs the transport of the βαIi complexes to endosomes and lysosomes. The proinflammatory cytokine, macrophage migration-inhibitory factor (MIF), binds to cell surface CD74, initiating a signaling cascade involving activation of NF-κB. CD74 is expressed by certain normal HLA class II-positive cells, including B cells, monocytes, macrophages, Langerhans cells, dendritic cells, subsets of activated T cells, and thymic epithelium. CD74 is also expressed on a variety of malignant cells, including the vast majority of B-cell cancers (NHL, CLL, MM). Expression of CD74 has been observed by DNA microarray-based methodology in AML clinical samples, and it has been shown to be a prognostic factor in the cytogenetically normal subset of AML, and to be a predictive factor for response to bortezomib in combination with induction chemotherapy.

The LL1 monoclonal antibody was generated by hybridoma technology after immunization of BALB/c mice with Raji human Burkitt lymphoma cells. The LL1 antibody reacts with an epitope in the extracellular domain of CD74. CD74-positive cell lines have been shown to very rapidly internalize LL1 (nearly 10⁷ molecules per cell per day). This rapid internalization enables LL1 to be an extremely effective agent for delivery of cytotoxic agents, such as chemotherapeutics or toxins, to malignant target cells.

Humanized anti-CD74 LL1 antibody (milatuzumab) exhibits direct cytotoxicity for NHL, CLL and MM cell lines, and is in clinical evaluation for therapy of NHL, MM and CLL. CD74 is induced by interferons in multiple cancer cell lines. Here we report an evaluation of CD74 expression and function in AML, and the effect of CD74 upregulation by treatment with IFN-γ on the cytotoxicity of milatuzumab for AML cell lines.

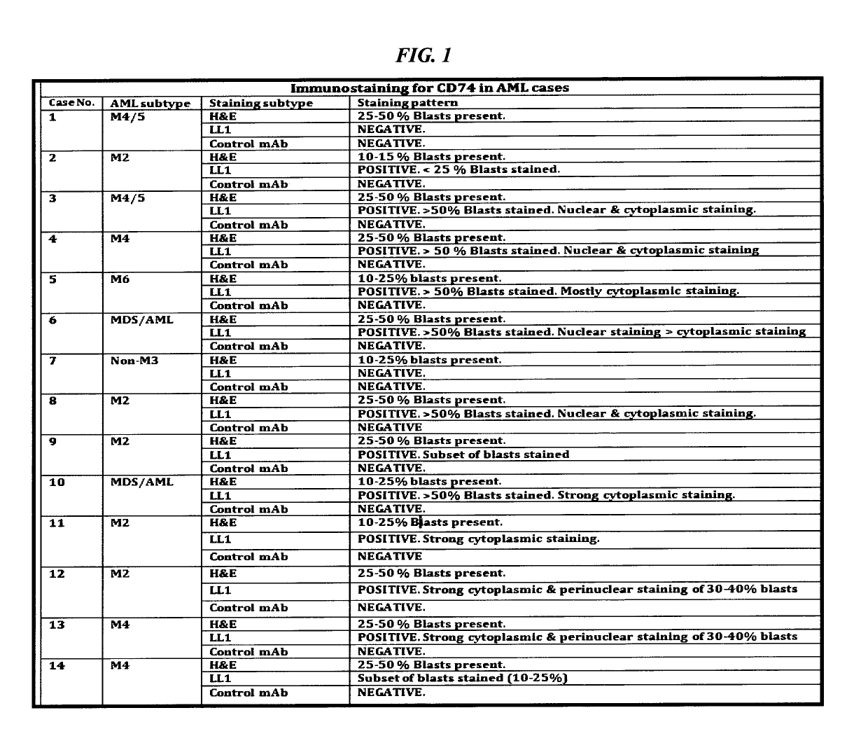

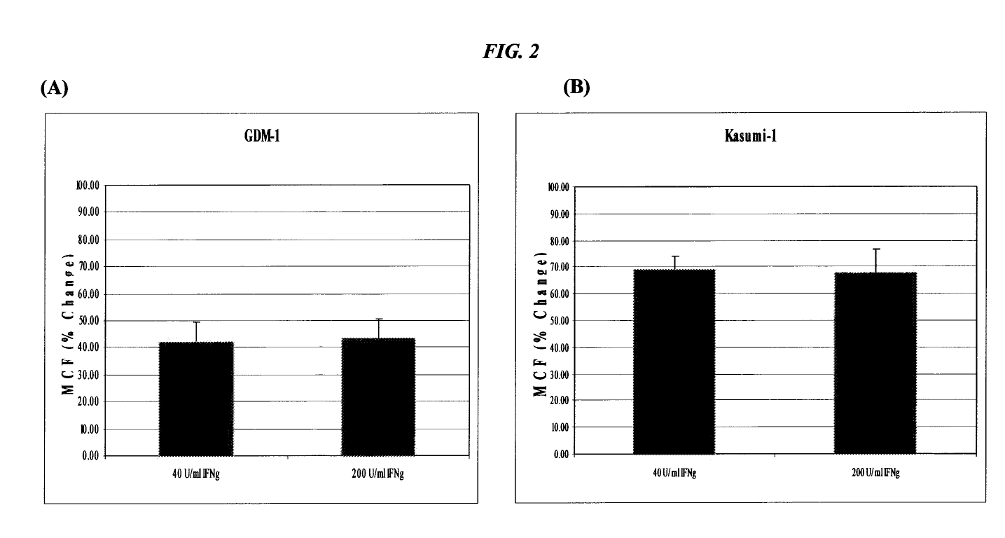

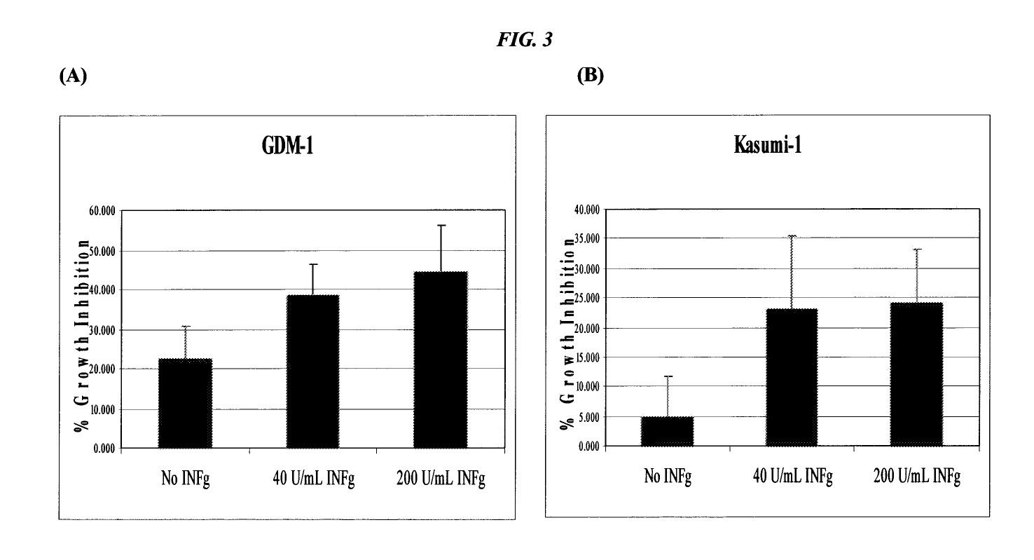

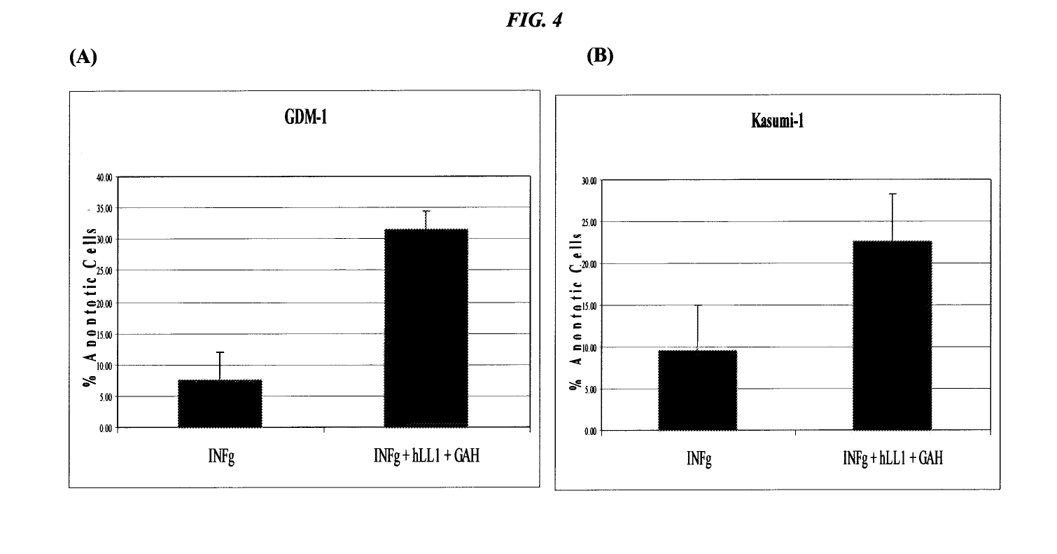

CD74 expression in bone marrow biopsy (BMB) specimens from non-M3 AML patients was evaluated by immunohistochemistry and, for 3 human AML cell lines, by flow cytometry, with/without permeabilization and with/without IFN-γ (40 and 200 U/mL). These cell lines were also tested in proliferation assays for responses to milatuzumab, with/without IFN-γ. In 13/14 BMB specimens (FIG. 1), there was moderate to strong CD74 expression by leukemic blasts, which was mostly intracellular, usually with a perinuclear distribution. Three AML cell lines also showed moderate to strong expression of CD74, which was mostly intracellular (data not shown). Without IFN-γ, surface expression of CD74 was present, but IFN-γ treatment of these 3 lines resulted in upregulation of surface CD74 by 69-117% (not shown). Much higher levels of intracellular CD74 were observed in all 3 lines, with and without IFN-γ (not shown). IFN-γ induced intracellular CD74 in all 3 lines (from 85%-868%) (see, e.g., FIG. 2A-2B). In 2/3 lines, IFN-γ increased milatuzumab-mediated growth inhibition (23.7 to 44.8% and −3.9 to 30.9%, respectively) (FIG. 3, FIG. 4).

CD74 is expressed in AML patient specimens and in AML cell lines, with the majority of CD74 expression found intracellularly. Cell surface and cytoplasmic expression of CD74 were upregulated in AML lines after IFN-γ exposure. This increased expression resulted in increased cytotoxicity of the anti-CD74 MAb, milatuzumab, in 2/3 AML lines. Thus, combined therapy with IFN-γ and milatuzumab treatment is of use for treatment of AML.

### Example 2

### Sensitivity of NHL to Killing by Anti-HLA-DR and Anti-CD74 Antibodies is Increased by Interferon-γ

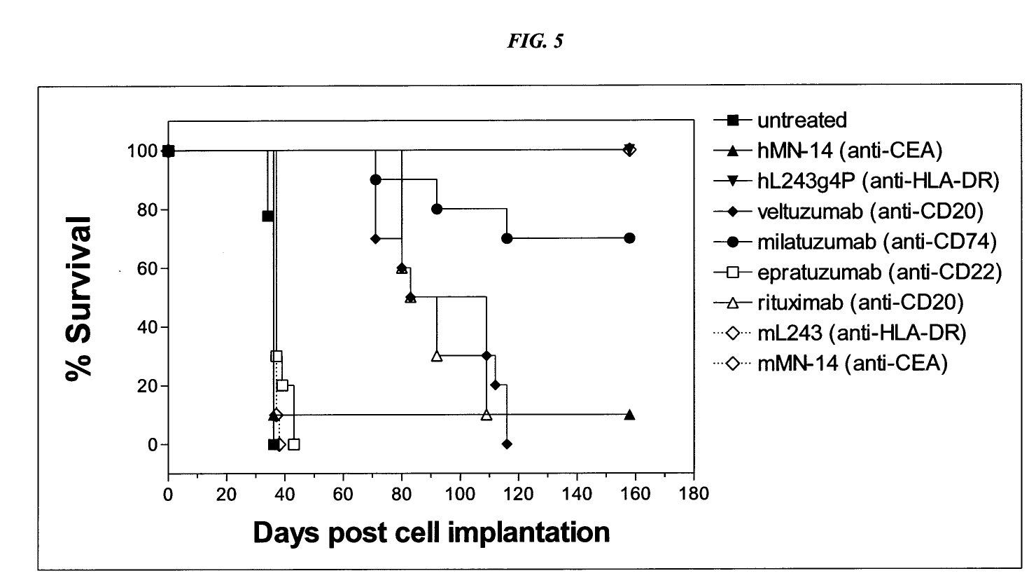

HLA-DR and CD74 are similarly, but not identically, expressed and induced by interferons on a variety of cells. Expression of both antigens on hematological malignancies led to their development as targets for antibody-based therapy. During our previous work on anti-B-cell MAbs, we observed that the anti-HLA-DR and anti-CD74 MAbs, hL243g4P and milatuzumab, had potent therapeutic activity toward B-cell malignancies. Milatuzumab is in clinical evaluation for therapy of NHL, multiple myeloma (MM), and CLL after preclinical evidence of activity in these tumor types, while clinical trials are planned for hL243g4P (IMMU-114). In representative data shown in FIG. 5, SCID mice bearing WSU-FSCCL follicular lymphoma are more sensitive to these two MAbs than to anti-CD20 MAbs such as rituximab.

In addition to expression in hematologic cancers, these antigens are expressed on the surface of other types of tumor cells, including melanoma and renal cell carcinoma, and in the cytoplasm of others, including pancreatic and colonic carcinomas and glioblastomas (GBM).

We examined whether the ability of anti-HLA-DR and anti-CD74 MAbs to kill cancer cells can be increased by using IFN-γ as an inducer of antigen expression. Using a panel of diverse cancer cell lines (including NHL, MM, GBM, and pancreatic and colonic carcinomas), we examined IFN-γ-induced changes in surface and cytoplasmic HLA-DR and CD74 expression. Sensitivity of the malignant cells to milatuzumab and hL243g4P was assessed with and without IFN-γ by cytotoxicity assays.

Results

Expression of CD74, HLA-DR, and carcinoembryonic antigen (CEACAM5) were determined in untreated cells and cells exposed to 200 U of IFN-γ for 48 h by flow cytometry. Cells were stained with directly labeled MAbs in comparison to a directly labeled human IgG control. Antibodies were labeled using ALEXA FLUOR® 488 (Invitrogen, Carlsbad, Calif.). For determination of cytoplasmic antigen expression, cells were permeabilized prior to staining using the BD CYTOFIX/CYTOPERM™ kit (BD Biosciences, San Jose, Calif.).

Without IFN-γ, surface expression of HLA-DR and CD74 was present on 2/2 NHL, 2/2 mM, and only weakly positive on 2/2 GBM cell lines (Table 2). Surface CD74 and HLA-DR were weak or undetectable on 4/4 colon and 4/4 pancreatic carcinomas (Table 2). Cytoplasmic

TABLE 2
Cell Surface Expression of CD74, HLA-DR and CEACAM5 With and Without IFN-γ

| | hIgG | Labetuzumab | hL243γ4P | Milatuzumab |
|---|---|---|---|---|
| | (control) | (anti-CEA) | (anti-HLA-DR) | (anti-CD74) |
| Lymphomas | | | | |
| FSCCL | 2.3 | 2.6 | 1087.1 | 14.3 |
| FSCCL + IFNγ | 2.8 | 2.8 | 1610.9 | 19.0 |
| % change | 25.8 | 7.6 | 48.2 | 32.8 |
| RL | 4.0 | 2.4 | 749.5 | 20.2 |
| RL + IFNγ | 2.8 | 2.9 | 861.8 | 25.0 |
| % change | −29.4 | 17.3 | 15.0 | 23.8 |
| Multiple Myelomas | | | | |
| CAG | 5.2 | 5.0 | 1926.3 | 41.5 |
| CAG + IFNγ | 5.4 | 5.2 | 1813.9 | 39.2 |
| % change | 2.9 | 3.0 | −5.8 | −5.4 |
| KMS11 | 2.3 | 2.1 | 677.2 | 4.4 |
| KMS11 + IFNγ | 2.5 | 2.3 | 665.3 | 5.1 |
| % change | 5.2 | 7.1 | −1.8 | 14.0 |
| Pancreatic Cancers | | | | |
| Panc-1 | 4.3 | 4.1 | 4.3 | 4.5 |
| Panc-1 + IFNγ | 4.2 | 4.4 | 4.7 | 4.9 |
| % change | −2.3 | 6.3 | 9.2 | 9.4 |
| Capan-1 | 4.2 | 57.5 | 5.5 | 4.4 |
| Capan + IFNγ | 5.3 | 48.8 | 66.2 | 11.1 |
| % change | 26.6 | −15.1 | 1100.0 | 152.4 |
| Aspc-1 | 3.0 | 52.9 | 3.2 | 3.3 |
| Aspc-1 + IFNγ | 3.4 | 66.8 | 15.2 | 7.3 |
| % change | 13.9 | 26.3 | 373.5 | 121.3 |
| BxPC-3 | 2.4 | 5.6 | 2.3 | 2.6 |
| BxPC-3 + IFNγ | 3.7 | 7.0 | 43.8 | 6.2 |
| % change | 55.7 | 25.6 | 1771.4 | 142.2 |
| Colon Cancers | | | | |
| Lovo | 3.1 | 56.8 | 7.3 | 3.4 |
| Lovo + IFNγ | 4.4 | 84.4 | 276.3 | 9.2 |
| % change | 45.1 | 48.6 | 3705.1 | 173.7 |
| Moser | 3.9 | 63.8 | 4.0 | 4.1 |
| Moser + IFNγ | 4.0 | 77.2 | 8.5 | 4.8 |
| % change | 2.3 | 20.9 | 113.0 | 16.3 |
| HT29 | 3.3 | 11.1 | 3.3 | 3.4 |
| HT29 + IFNγ | 4.8 | 34.9 | 298.0 | 8.3 |
| % change | 45.5 | 213.5 | 8848.9 | 141.1 |
| LS174T | 4.8 | 61.1 | 5.3 | 5.9 |
| LS174T + IFNγ | 4.8 | 163.7 | 4.7 | 5.2 |
| % change | 0.0 | 167.9 | −11.7 | −11.3 |
| Glioblastomas | | | | |
| U87 | 3.3 | 3.7 | 41.9 | 5.4 |
| U87 + IFNγ | 3.3 | 4.5 | 171.5 | 9.5 |
| % change | −0.3 | 23.8 | 309.0 | 75.6 |
| U118 | 4.2 | 5.5 | 6.1 | 7.0 |
| U118 + IFNγ | 4.5 | 5.6 | 197.1 | 18.3 |
| % change | 7.2 | 2.4 | 3136.9 | 160.9 |
| TU118 | 4.72 | 4.68 | 5.08 | 7.17 |
| TU118 + IFNγ | 5.61 | 5.29 | 93.49 | 19.2 |
| % change | 18.9 | 13.0 | 1740.4 | 167.8 |

TABLE 3
Cytoplasmic Expression of CD74, HLA-DR and CEACAM5 With and Without IFN-γ

| | hIgG | Labetuzumab | hL243γ4P (anti- | Milatuzumab |
|---|---|---|---|---|
| | (control) | (anti-CEA) | HLA-DR) | (anti-CD74) |
| Lymphomas | | | | |
| FSCCL | 27.3 | 19.3 | 1466.8 | 708.0 |
| FSCCL + IFNγ | 45.3 | 38.8 | 2522.2 | 1122.0 |
| % change | 66.0 | 100.7 | 72.0 | 58.5 |
| RL | 13.4 | 7.8 | 1055.3 | 887.8 |
| RL + IFNγ | 16.4 | 10.8 | 1184.3 | 920.9 |
| % change | 22.6 | 37.9 | 12.2 | 3.7 |
| Multiple Myelomas | | | | |
| CAG | 10.0 | 7.1 | 2315.2 | 418.1 |
| CAG + IFNγ | 12.4 | 9.1 | 2422.9 | 501.0 |
| % change | 24.6 | 27.4 | 4.7 | 19.8 |
| KMS11 | 10.5 | 8.1 | 878.6 | 228.8 |
| KMS11 + IFNγ | 11.7 | 7.2 | 926.8 | 224.5 |
| % change | 12.0 | −11.4 | 5.5 | −1.9 |
| Pancreatic Cancers | | | | |
| Panc-1 | 22.8 | 20.3 | 22.7 | 24.4 |
| Panc-1 + IFNγ | 56.0 | 53.0 | 64.1 | 75.3 |
| % change | 146.0 | 161.2 | 181.9 | 208.7 |
| Capan-1 | 12.4 | 168.2 | 11.6 | 41.2 |
| Capan + IFNγ | 16.9 | 182.7 | 253.1 | 272.1 |
| % change | 36.0 | 8.7 | 2081.6 | 561.2 |
| Aspc-1 | 13.5 | 139.0 | 11.0 | 12.8 |
| Aspc-1 + IFNγ | 31.0 | 213.0 | 73.6 | 198.4 |
| % change | 130.0 | 53.2 | 571.9 | 1451.5 |
| BxPC-3 | 18.4 | 22.2 | 14.2 | 15.3 |
| BxPC-3 + IFNγ | 27.1 | 33.9 | 129.7 | 285.5 |
| % change | 47.0 | 52.7 | 811.0 | 1763.5 |
| Colon Cancers | | | | |
| Lovo | 22.0 | 127.2 | 32.5 | 39.7 |
| Lovo + IFNγ | 41.4 | 193.8 | 989.3 | 339.7 |
| % change | 88.2 | 52.4 | 2942.0 | 756.0 |
| Moser | 24.5 | 102.6 | 18.2 | 28.9 |
| Moser + IFNγ | 36.7 | 145.5 | 40.5 | 53.7 |
| % change | 49.5 | 41.8 | 122.1 | 86.1 |
| HT29 | 9.6 | 35.9 | 9.1 | 10.8 |
| HT29 + IFNγ | 22.9 | 80.0 | 638.1 | 202.0 |
| % change | 139.1 | 122.7 | 6919.8 | 1766.6 |
| LS174T | 29.4 | 154.4 | 23.4 | 34.6 |
| LS174T + IFNγ | 51.2 | 456.9 | 42.3 | 73.6 |
| % change | 74.2 | 195.9 | 81.0 | 112.7 |
| Glioblastomas | | | | |
| U87 | 5.4 | 11.9 | 102.4 | 54.9 |
| U87 + IFNγ | 5.5 | 21.3 | 429.0 | 141.9 |
| % change | 2.6 | 79.0 | 318.9 | 158.8 |
| U118 | 7.7 | 17.1 | 24.6 | 47.7 |
| U118 + IFNγ | 7.5 | 30.5 | 352.3 | 252.1 |
| % change | −2.5 | 78.2 | 1333.4 | 428.2 |
| TU118 | 35.11 | 20.4 | 23.61 | 121.92 |
| TU118 + IFNγ | 57.21 | 43.96 | 215.38 | 578.58 |
| % change | 62.9 | 115.5 | 812.2 | 374.6 |

CD74 and HLA-DR were seen in the NHL, MM, GBM, and 1/4 colon and 1/4 pancreatic (CD74 only) carcinomas (Table 3).

Two-day incubation with IFN-γ increased surface and cytoplasmic expression of CD74 and HLA-DR (Table 2, Table 3). In all 4 colon cancer lines, IFN-γ increased cytoplasmic expression of both antigens, and surface expression of HLA-DR in 3/4 and CD74 in 2/4 (Table 2, Table 3). Upregulation of HLA-DR and CD74 ranged from 23-3700% (Table 2, Table 3).

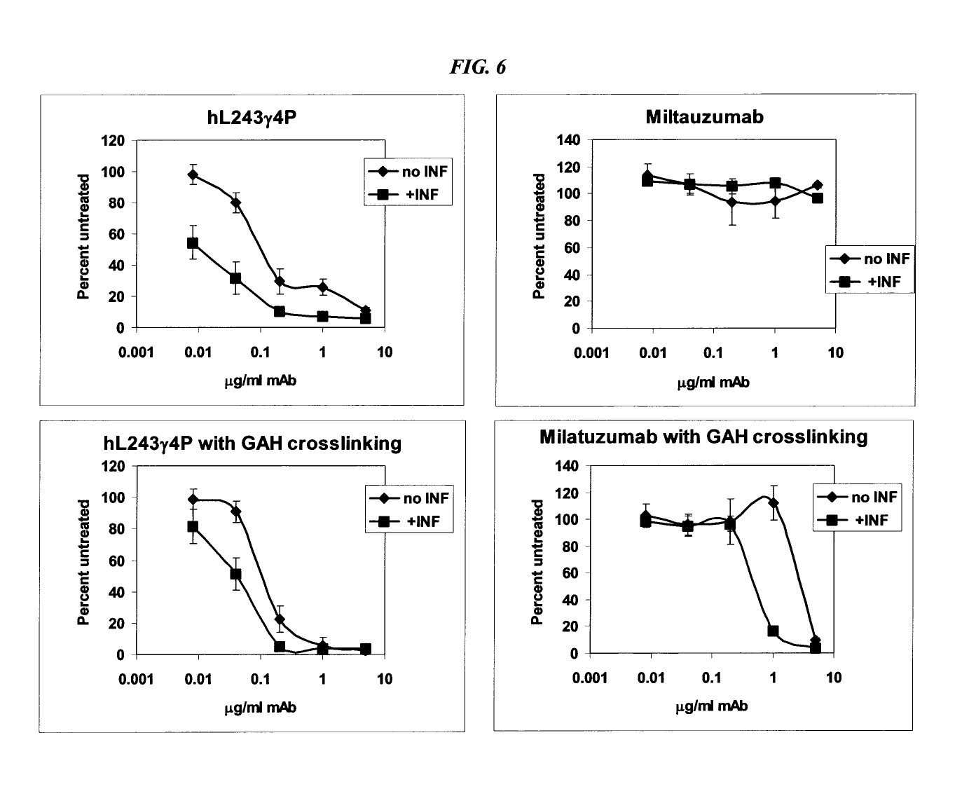

The cytotoxicity of anti-CD74 and anti-HLA-DR antibodies was examined in the presence or absence of IFN-γ (FIG. 6). As previously observed, in vitro cytotoxicity of milatuzumab, but not hL243g4P, required crosslinking (Stein, et al., Blood, 104: 3705-11, 2004) (FIG. 6). Goat anti-human IgG (GAH) was used for crosslinking in these experiments. Increased killing by both hL243g4P (58%) and milatuzumab (33%) was seen in vitro after IFN-γ exposure in WSU-FSCCL NHL cells (FIG. 6). Cytotoxicity was in part due to apoptosis, as significant increases in Annexin V binding (P=0.01) were observed after treatment with IFN-γ plus milatuzumab (not shown). Experiments addressing cell signaling suggest a role for AKT (EXAMPLE 3), since phosphorylated AKT levels increased (P=0.06) in response to IFN-γ+milatuzumab (not shown). Milatuzumab and hL243g4P were unable to kill Capan-1 (pancreatic carcinoma), Aspc-1 (pancreatic carcinoma), LoVo (colon carcinoma), HT-29 (colon carcinoma), U87 (GBM), or U118 (GBM) cells, regardless of the use of a crosslinking agent or IFN-γ-induced upregulation of antigen expression.

Conclusions

Cell surface and cytoplasmic expression of CD74 and HLA-DR were increased on cell lines from a variety of cancer types after IFN-γ exposure. In the follicular lymphoma cell line, WSU-FSCCL, the increased expression of these antigens correlates with increased toxicity of hL243g4P and milatuzumab. These studies demonstrate the potential benefit of combined IFN-γ and anti-CD74 and/or anti-HLA-DR antibody therapies.

### Example 3

### Effect of Anti-HLA-DR Antibody is Mediated Through ERK and JNK MAP Kinase Signaling Pathways

We examined the reactivity and cytotoxicity of the humanized anti-HLA-DR antibody hL243γ4P (IMMU-114) on a panel of leukemia cell lines. hL243γ4P bound to the cell surface of 2/3 AML, 2/2 mantle cell, 4/4 ALL, 1/1 hairy cell leukemia, and 2/2 CLL cell lines, but not on the 1 CML cell line tested (not shown). Cytotoxicity assays demonstrated that hL243γ4P was toxic to 2/2 mantle cell, 2/2 CLL, 3/4 ALL, and 1/1 hairy cell leukemia cell lines, but did not kill 3/3 AML cell lines despite positive staining (not shown). As expected, the CML cell line was also not killed by hL243γ4P (not shown).

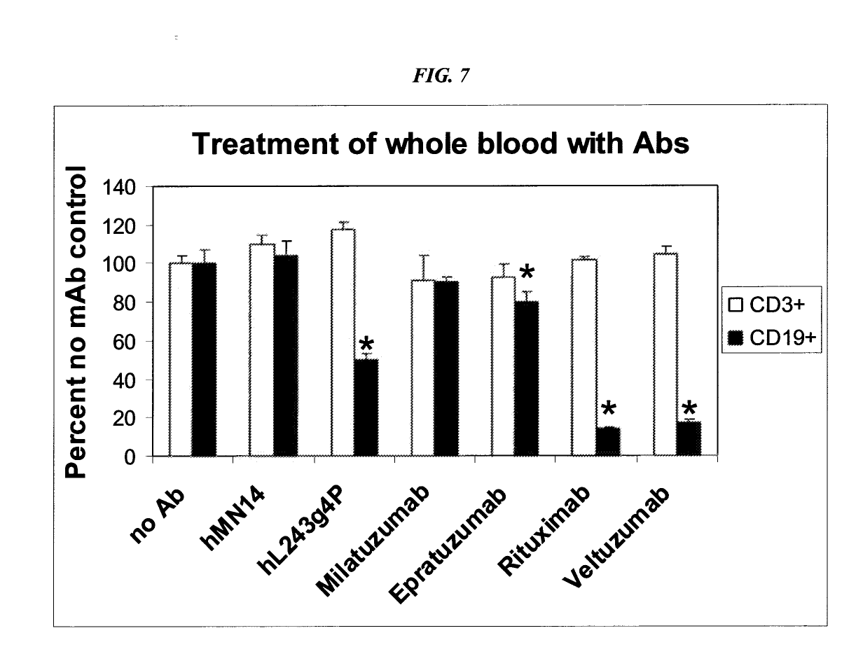

FIG. 7 illustrates the ex vivo effects of various antibodies on whole blood. hL243γ4P resulted in significantly less B cell depletion than rituximab and veltuzumab, consistent with an earlier report (Nagy, et al, J Mol Med 2003; 81:757-65) which suggested that anti-HLA-DR MAbs kill activated, but not resting normal B cells, in addition to tumor cells. This suggests a dual requirement for both MHC-II expression and cell activation for antibody-induced death, and implies that because the majority of peripheral B cells are resting, the potential side effect due to killing of normal B cells may be minimal. T-cells are unaffected.

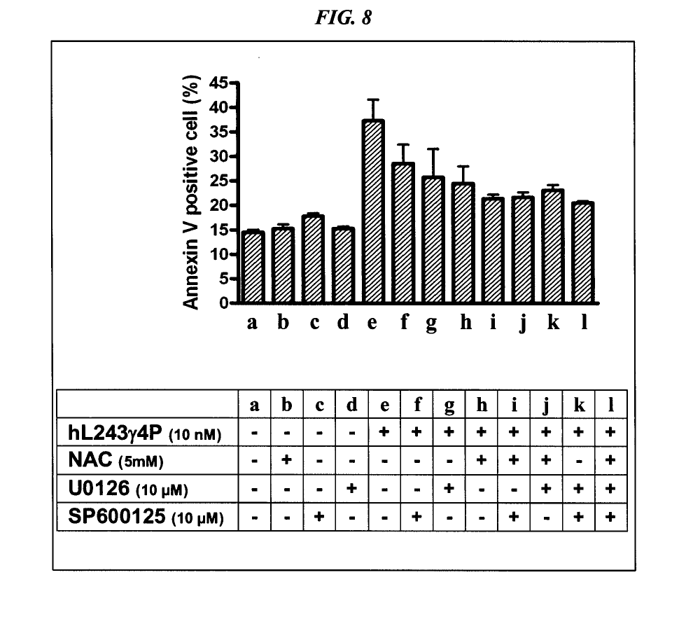

The effects of ERK, JNK and ROS inhibitors on hL243γ4P mediated apoptosis in Raji cells is shown in FIG. 8. hL243γ4P cytotoxicity correlates with activation of ERK and JNK signaling and differentiates the mechanism of action of hL243γ4P cytotoxicity from that of anti-CD20 MAbs. hL243γ4P also changes mitochondrial membrane potential and generates ROS in Raji cells (not shown). Inhibition of ERK, JNK, or ROS by specific inhibitors partially abrogates the apoptosis. Inhibition of 2 or more pathways abolishes the apoptosis.

Signaling pathways were studied to elucidate why cytotoxicity does not always correlate with antigen expression in the malignant B-cell lines examined. Various pathways were compared in IMMU-114-sensitive and -resistant HLA-DR-expressing cell lines. The AML lines, Kasumi-3 and GDM-1, were used as examples of HLA-DR⁺ cell lines resistant to IMMU-114 cytotoxicity. IMMU-114-sensitive cells included NHL (Raji), MCL (Jeko-1 and Granta-519), CLL (WAC and MEC-1), and ALL (REH and MN60). Results of Western blot analyses of these cell lines revealed that IMMU-114 induces phosphorylation and activation of ERK and JNK mitogen activated protein (MAP) kinases in all the cells defined as IMMU-114-sensitive by the cytotoxicity assays, but not the IMMU-114-resistant cell lines, Kasumi-3 and GDM-1 (data not shown). p38 MAP kinase was found to be constitutively active in these cell lines, and no further activation beyond basal levels was noted (data not shown).

Two methods were used to confirm the importance of the ERK and JNK signaling pathways in the IMMU-114 mechanism of action. These involved use of specific chemical inhibitors of these pathways and siRNA inhibition. ERK, JNK, and ROS inhibitors used were: NAC (5 mM) blocks ROS, U0126 (10 μM) blocks MEK phosphorylation and the ERK1/2 pathway, and SP600125 (10 μM) blocks the JNK pathway. Inhibition of ERK, JNK, or ROS by their respective inhibitors decreased apoptosis in Raji cells, although the inhibition was not complete when any single inhibitor was used (not shown). This may have been the result of activation of multiple pathways because inhibition of 2 or more pathways by specific inhibitors abolished the IMMU-114-induced apoptosis (not shown). Transfection of Raji cells with siERK and siJNK RNAs effectively lowered the expression of ERK and JNK proteins and significantly inhibited IMMU-114-induced apoptosis (not shown) validating the role of these pathways in IMMU-114 cell killing.

The AML lines, Kasumi-3 and GDM-1, were resistant to apoptosis mediated by IMMU-114 (as measured by annexin V, data not shown). Significant changes in mitochondrial membrane potential and generation of ROS also were not observed on treatment of these AML cell lines with IMMU-114 (not shown). Sensitive lines, such as Raji, showed a greater degree of homotypic aggregation on treatment with IMMU-114, whereas aggregation was not observed in AML lines, such as Kasumi-3 (data not shown).

Activation of ERK1/2 and JNK signaling pathways was also assessed in CLL patient samples (not shown). Patient cells were incubated with IMMU-114 for 4 hours because the cells in these samples were much smaller than those of the established cell lines. Moreover, the shorter incubation time avoids the risk of higher apoptosis and cell death. Similar to our observations in the IMMU-114-sensitive cell lines, activation and phosphorylation of the ERK1/2 and JNK pathways were observed in the CLL patient cells, indicating the generation of stress in these samples (not shown). Almost 4- to 5-fold activation of ERK and JNK pathways was observed on incubation with IMMU-114 over untreated controls, although no such activation was seen on treatment with rituximab or milatuzumab (not shown).

To further investigate the molecular mechanism whereby IMMU-114 induces cell death, we investigated the effect of IMMU-114 on changes in mitochondrial membrane potential and production of ROS. Treatment with IMMU-114 induced a time-dependent mitochondrial membrane depolarization that could be detected in Raji cells as well as in other sensitive lines (not shown). A time-course analysis in Raji cells indicated a change in mitochondrial membrane depolarization of 46% in as little as 30 minutes of treatment, and a further increase to 66% in 24 hours (not shown). Similar changes in ROS levels were observed (not shown). A thirty minute incubation with IMMU-114 induced a 24% change in ROS levels that increased to 33% to 44% on overnight incubation (not shown). Preincubation of Raji cells with the ROS inhibitor NAC blocked the generation of ROS on treatment with IMMU-114; only 8% ROS was observed in IMMU-114 plus NAC-treated cells (not shown). Changes in mitochondrial membrane potential were also abrogated by the ROS inhibitor (not shown). These observations suggest that ROS generation plays a crucial role in IMMU-114-induced cell death and are consistent with the action of IMMU-114 on ROS being an early effect occurring before apoptosis.

Discussion

To characterize the cytotoxic mechanism of IMMU-114, we compared the activation of ERK, JNK, and p38 MAP kinases in our panel of cell lines and CLL patient cells. We found that JNK1/2 and ERK1/2 phosphorylation was up-regulated after exposure of cells to IMMU-114 in sensitive cell lines, such as the CLL patient cells, and the Raji and Jeko-1 cell lines, but not in the IMMU-114-resistant AML cell lines, such as Kasumi-3 and GDM-1. We observed up to 5-fold activation of the ERK and JNK signaling pathways on treatment with IMMU-114 at a modest 10-nM concentration. p38 MAP kinase was found to be constitutively active in these cell lines, and no further activation beyond basal levels was noted. Inhibition of the ERK and JNK signaling cascades by their respective inhibitors could modestly inhibit the apoptosis induced by IMMU-114. However, apoptosis was completely inhibited when 2 inhibitors were used together, indicating the activation of multiple MAP kinases by IMMU-114. IMMU-114-induced apoptosis was also significantly inhibited by siERK and siJNK RNAs. Thus, IMMU-114 cytotoxicity correlates with activation of ERK and JNK signaling. In addition, the results of these studies differentiate the mechanism of action of IMMU-114 cytotoxicity from that of the anti-CD74 (milatuzumab) and anti-CD20 MAbs.

Contemporary cancer drug development is focused on “targeted” therapies, which includes agents that selectively attack a survival pathway for cancer cells. Antibodies that can perform this function are of great interest. The anti-HLA-DR MAb, IMMU-114, an agent that reacts with a variety of hematologic malignancies, is one of the most effective therapeutic MAbs that we have examined and shows cytotoxicity in rituximab-resistant NHL cell lines. Variation in expression and cytotoxicity profiles between the MAbs suggests that combination therapies may yield greater effects in these various malignancies than the MAbs given singly, as reported previously in NHL cell lines.

### Example 4

### Preparation of Dock-and-Lock (DNL) Constructs

DDD and AD Fusion Proteins

The DNL technique can be used to make dimers, trimers, tetramers, hexamers, etc. comprising virtually any antibody, antibody fragment, cytokine or other effector moiety. For certain preferred embodiments, antibodies and cytokines may be produced as fusion proteins comprising either a dimerization and docking domain (DDD) or anchoring domain (AD) sequence. Although in preferred embodiments the DDD and AD moieties may be joined to antibodies, antibody fragments or cytokines as fusion proteins, the skilled artisan will realize that other methods of conjugation exist, such as chemical cross-linking, click chemistry reaction, etc.

The technique is not limiting and any protein or peptide of use may be produced as an AD or DDD fusion protein for incorporation into a DNL construct. Where chemical cross-linking is utilized, the AD and DDD conjugates may comprise any molecule that may be cross-linked to an AD or DDD sequence using any cross-linking technique known in the art. In certain exemplary embodiments, a dendrimer or other polymeric moiety such as polyethyleneimine or polyethylene glycol (PEG), may be incorporated into a DNL construct, as described in further detail below.

For different types of DNL constructs, different AD or DDD sequences may be utilized. Exemplary DDD and AD sequences are provided below.

DDD1:
(SEQ ID NO: 45)
SHIQIPPGLTELLQGYTVEVLRQQPPDLVEFAVEYFTRLREARA
DDD2:
(SEQ ID NO: 46)
CGHIQIPPGLTELLQGYTVEVLRQQPPDLVEFAVEYFTRLREARA
AD1:
(SEQ ID NO: 47)
QIEYLAKQIVDNAIQQA
AD2:
(SEQ ID NO: 48)
CGQIEYLAKQIVDNAIQQAGC

The skilled artisan will realize that DDD1 and DDD2 comprise the DDD sequence of the human riiα form of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 49)
SLRECELYVQKHNIQALLKDSIVQLCTARPERPMAFLREYFERLEKEEAK
DDD3C
(SEQ ID NO: 50)
MSCGGSLRECELYVQKHNIQALLKDSIVQLCTARPERPMAFLREYFERLEKEEAK
AD3
(SEQ ID NO: 51)
CGFEELAWKIAKMIWSDVFQQGC

Expression Vectors

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH2 and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445-50.

Two shuttle vectors were designed to facilitate the conversion of IgG-pdHL2 vectors to either Fab-DDD1 or Fab-AD1 expression vectors, as described below.

Preparation of CH1

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge (PKSC) followed by four glycines and a serine, with the final two codons (GS) comprising a Bam HI restriction site. The 410 by PCR amplimer was cloned into the PGEMT® PCR cloning vector (PROMEGA®, Inc.) and clones were screened for inserts in the T7 (5′) orientation.

A duplex oligonucleotide was synthesized to code for the amino acid sequence of DDD1 preceded by 11 residues of the linker peptide, with the first two codons comprising a BamHI restriction site. A stop codon and an EagI restriction site are appended to the 3′ end. The encoded polypeptide sequence is shown below.

(SEQ ID NO: 52)
GSGGGGSGGGGSHIQIPPGLTELLQGYTVEVLRQQPPDLVEFAVEYFTRLREARA

Two oligonucleotides, designated RIIA1-44 top and RIIA1-44 bottom, which overlap by 30 base pairs on their 3′ ends, were synthesized and combined to comprise the central 154 base pairs of the 174 by DDD1 sequence. The oligonucleotides were annealed and subjected to a primer extension reaction with Taq polymerase. Following primer extension, the duplex was amplified by PCR. The amplimer was cloned into PGEMT® and screened for inserts in the T7 (5′) orientation.

A duplex oligonucleotide was synthesized to code for the amino acid sequence of AD1 preceded by 11 residues of the linker peptide with the first two codons comprising a BamHI restriction site. A stop codon and an EagI restriction site are appended to the 3′ end. The encoded polypeptide sequence is shown below.

(SEQ ID NO: 53)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the PGEMT® vector and screened for inserts in the T7 (5′) orientation.

Ligating DDD1 with CH1

A 190 bp fragment encoding the DDD1 sequence was excised from PGEMT® with BamHI and NotI restriction enzymes and then ligated into the same sites in CH1-PGEMT® to generate the shuttle vector CH1-DDD1-PGEMT®.

Ligating AD1 with CH1

A 110 bp fragment containing the AD1 sequence was excised from PGEMT® with BamHI and NotI and then ligated into the same sites in CH1-PGEMT® to generate the shuttle vector CH1-AD1-PGEMT®.

Cloning CH1-DDD1 or CH1-AD1 into pdHL2-Based Vectors

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective pGemT shuttle vector.

Construction of h679-Fd-AD1-pdHL2

h679-Fd-ADI-pdHL2 is an expression vector for production of h679 Fab with AD1 coupled to the carboxyl terminal end of the CH1 domain of the Fd via a flexible Gly/Ser peptide spacer composed of 14 amino acid residues. A pdHL2-based vector containing the variable domains of h679 was converted to h679-Fd-AD1-pdHL2 by replacement of the SacII/EagI fragment with the CH1-AD1 fragment, which was excised from the CH1-AD1-SV3 shuttle vector with SacII and EagI.

Construction of C-DDD1-Fd-hMN-14-pdHL2

C-DDD1-Fd-hMN-14-pdHL2 is an expression vector for production of a stable dimer that comprises two copies of a fusion protein C-DDD1-Fab-hMN-14, in which DDD1 is linked to hMN-14 Fab at the carboxyl terminus of CH1 via a flexible peptide spacer. The plasmid vector hMN-14(I)-pdHL2, which has been used to produce hMN-14 IgG, was converted to C-DDD1-Fd-hMN-14-pdHL2 by digestion with SacII and EagI restriction endonucleases to remove the CH1-CH3 domains and insertion of the CH1-DDD1 fragment, which was excised from the CH1-DDD1-SV3 shuttle vector with SacII and EagI.

The same technique has been utilized to produce plasmids for Fab expression of a wide variety of known antibodies, such as hLL1, hLL2, hPAM4, hR1, hRS7, hMN-15, hA19, hA20 and many others. Generally, the antibody variable region coding sequences were present in a pdHL2 expression vector and the expression vector was converted for production of an AD- or DDD-fusion protein as described above. The AD- and DDD-fusion proteins comprising a Fab fragment of any of such antibodies may be combined, in an approximate ratio of two DDD-fusion proteins per one AD-fusion protein, to generate a trimeric DNL construct comprising two Fab fragments of a first antibody and one Fab fragment of a second antibody.

Construction of N-DDD1-Fd-hMN-14-pdHL2

N-DDD1-Fd-hMN-14-pdHL2 is an expression vector for production of a stable dimer that comprises two copies of a fusion protein N-DDD1-Fab-hMN-14, in which DDD1 is linked to hMN-14 Fab at the amino terminus of VH via a flexible peptide spacer. The expression vector was engineered as follows. The DDD1 domain was amplified by PCR.

As a result of the PCR, an NcoI restriction site and the coding sequence for part of the linker containing a BamHI restriction were appended to the 5′ and 3′ ends, respectively. The 170 bp PCR amplimer was cloned into the pGemT vector and clones were screened for inserts in the T7 (5′) orientation. The 194 bp insert was excised from the pGemT vector with NcoI and Sail restriction enzymes and cloned into the SV3 shuttle vector, which was prepared by digestion with those same enzymes, to generate the intermediate vector DDD1-SV3.

The hMN-14 Fd sequence was amplified by PCR. As a result of the PCR, a BamHI restriction site and the coding sequence for part of the linker were appended to the 5′ end of the amplimer. A stop codon and EagI restriction site was appended to the 3′ end. The 1043 bp amplimer was cloned into pGemT. The hMN-14-Fd insert was excised from pGemT with BamHI and EagI restriction enzymes and then ligated with DDD1-SV3 vector, which was prepared by digestion with those same enzymes, to generate the construct N-DDD1-hMN-14Fd-SV3.

The N-DDD1-hMN-14 Fd sequence was excised with XhoI and EagI restriction enzymes and the 1.28 kb insert fragment was ligated with a vector fragment that was prepared by digestion of C-hMN-14-pdHL2 with those same enzymes. The final expression vector was N-DDD1-Fd-hMN-14-pDHL2. The N-linked Fab fragment exhibited similar DNL complex formation and antigen binding characteristics as the C-linked Fab fragment (not shown).

C-DDD2-Fd-hMN-14-pdHL2

C-DDD2-Fd-hMN-14-pdHL2 is an expression vector for production of C-DDD2-Fab-hMN-14, which possesses a dimerization and docking domain sequence of DDD2 appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

The expression vector was engineered as follows. Two overlapping, complimentary oligonucleotides, which comprise the coding sequence for part of the linker peptide and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-PGEMT®, which was prepared by digestion with BamHI and PstI, to generate the shuttle vector CH1-DDD2-PGEMT®. A 507 bp fragment was excised from CH1-DDD2-PGEMT® with SacII and EagI and ligated with the IgG expression vector hMN-14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

h679-Fd-AD2-pdHL2

h679-Fab-AD2, was designed to pair as B to C-DDD2-Fab-hMN-14 as A. h679-Fd-AD2-pdHL2 is an expression vector for the production of h679-Fab-AD2, which possesses an anchoring domain sequence of AD2 appended to the carboxyl terminal end of the CH1 domain via a 14 amino acid residue Gly/Ser peptide linker. AD2 has one cysteine residue preceding and another one following the anchor domain sequence of AD1.

The expression vector was engineered as follows. Two overlapping, complimentary oligonucleotides (AD2 Top and AD2 Bottom), which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-PGEMT®, which was prepared by digestion with BamHI and SpeI, to generate the shuttle vector CH1-AD2-PGEMT®. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

### Example 5

### Generation of TF1 DNL Construct

A large scale preparation of a DNL construct, referred to as TF1, was carried out as follows. N-DDD2-Fab-hMN-14 (Protein L-purified) and h679-Fab-AD2 (IMP-291-purified) were first mixed in roughly stoichiometric concentrations in 1 mM EDTA, PBS, pH 7.4. Before the addition of TCEP, SE-HPLC did not show any evidence of a₂b formation (not shown). Instead there were peaks representing a₄ (7.97 min; 200 kDa), a₂ (8.91 min; 100 kDa) and B (10.01 min; 50 kDa). Addition of 5 mM TCEP rapidly resulted in the formation of the a₂b complex as demonstrated by a new peak at 8.43 min, consistent with a 150 kDa protein (not shown). Apparently there was excess B in this experiment as a peak attributed to h679-Fab-AD2 (9.72 min) was still evident yet no apparent peak corresponding to either a₂ or a₄ was observed. After reduction for one hour, the TCEP was removed by overnight dialysis against several changes of PBS. The resulting solution was brought to 10% DMSO and held overnight at room temperature.

When analyzed by SE-HPLC, the peak representing a₂b appeared to be sharper with a slight reduction of the retention time by 0.1 min to 8.31 min (not shown), which, based on our previous findings, indicates an increase in binding affinity. The complex was further purified by IMP-291 affinity chromatography to remove the kappa chain contaminants. As expected, the excess h679-AD2 was co-purified and later removed by preparative SE-HPLC (not shown).

TF1 is a highly stable complex. When TF1 was tested for binding to an HSG (IMP-239) sensorchip, there was no apparent decrease of the observed response at the end of sample injection. In contrast, when a solution containing an equimolar mixture of both C-DDD1-Fab-hMN-14 and h679-Fab-AD1 was tested under similar conditions, the observed increase in response units was accompanied by a detectable drop during and immediately after sample injection, indicating that the initially formed a₂b structure was unstable. Moreover, whereas subsequent injection of WI2 gave a substantial increase in response units for TF1, no increase was evident for the C-DDD1/AD1 mixture.

The additional increase of response units resulting from the binding of WI2 to TF1 immobilized on the sensorchip corresponds to two fully functional binding sites, each contributed by one subunit of N-DDD2-Fab-hMN-14. This was confirmed by the ability of TF1 to bind two Fab fragments of WI2 (not shown). When a mixture containing h679-AD2 and N-DDD1-hMN14, which had been reduced and oxidized exactly as TF1, was analyzed by BIAcore, there was little additional binding of WI2 (not shown), indicating that a disulfide-stabilized a₂b complex such as TF1 could only form through the interaction of DDD2 and AD2.

Two improvements to the process were implemented to reduce the time and efficiency of the process. First, a slight molar excess of N-DDD2-Fab-hMN-14 present as a mixture of a₄/a₂ structures was used to react with h679-Fab-AD2 so that no free h679-Fab-AD2 remained and any a₄/a₂ structures not tethered to h679-Fab-AD2, as well as light chains, would be removed by IMP-291 affinity chromatography. Second, hydrophobic interaction chromatography (HIC) has replaced dialysis or diafiltration as a means to remove TCEP following reduction, which would not only shorten the process time but also add a potential viral removing step. N-DDD2-Fab-hMN-14 and 679-Fab-AD2 were mixed and reduced with 5 mM TCEP for 1 hour at room temperature. The solution was brought to 0.75 M ammonium sulfate and then loaded onto a Butyl FF HIC column. The column was washed with 0.75 M ammonium sulfate, 5 mM EDTA, PBS to remove TCEP. The reduced proteins were eluted from the HIC column with PBS and brought to 10% DMSO. Following incubation at room temperature overnight, highly purified TF1 was isolated by IMP-291 affinity chromatography (not shown). No additional purification steps, such as gel filtration, were required.

### Example 6

### Generation of TF2 DNL Construct

A trimeric DNL construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

The functionality of TF2 was determined by BIACORE® assay. TF2, C-DDD1-hMN-14+h679-AD1 (used as a control sample of noncovalent a₂b complex), or C-DDD2-hMN-14+h679-AD2 (used as a control sample of unreduced a₂ and b components) were diluted to 1 μg/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of WI2 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of WI2 to TF2 immobilized on the sensorchip corresponded to two fully functional binding sites, each contributed by one subunit of C-DDD2-Fab-hMN-14. This was confirmed by the ability of TF2 to bind two Fab fragments of WI2 (not shown).

### Example 7

### Production of AD- and DDD-Linked Fab and IgG Fusion Proteins From Multiple Antibodies

Using the techniques described in the preceding Examples, the IgG and Fab fusion proteins shown in Table 4 were constructed and incorporated into DNL constructs. The fusion proteins retained the antigen-binding characteristics of the parent antibodies and the DNL constructs exhibited the antigen-binding activities of the incorporated antibodies or antibody fragments.

TABLE 4
Fusion proteins comprising IgG or Fab

| Fusion Protein | Binding Specificity |
|---|---|
| C-AD1-Fab-h679 | HSG |
| C-AD2-Fab-h679 | HSG |
| C-(AD)₂-Fab-h679 | HSG |
| C-AD2-Fab-h734 | Indium-DTPA |
| C-AD2-Fab-hA20 | CD20 |
| C-AD2-Fab-hA20L | CD20 |
| C-AD2-Fab-hL243 | HLA-DR |
| C-AD2-Fab-hLL2 | CD22 |
| N-AD2-Fab-hLL2 | CD22 |
| C-AD2-IgG-hMN-14 | CEACAM5 |
| C-AD2-IgG-hR1 | IGF-1R |
| C-AD2-IgG-hRS7 | EGP-1 |
| C-AD2-IgG-hPAM4 | MUC |
| C-AD2-IgG-hLL1 | CD74 |
| C-DDD1-Fab-hMN-14 | CEACAM5 |
| C-DDD2-Fab-hMN-14 | CEACAM5 |
| C-DDD2-Fab-h679 | HSG |
| C-DDD2-Fab-hA19 | CD19 |
| C-DDD2-Fab-hA20 | CD20 |
| C-DDD2-Fab-hAFP | AFP |
| C-DDD2-Fab-hL243 | HLA-DR |
| C-DDD2-Fab-hLL1 | CD74 |
| C-DDD2-Fab-hLL2 | CD22 |
| C-DDD2-Fab-hMN-3 | CEACAM6 |
| C-DDD2-Fab-hMN-15 | CEACAM6 |
| C-DDD2-Fab-hPAM4 | MUC |
| C-DDD2-Fab-hR1 | IGF-1R |
| C-DDD2-Fab-hRS7 | EGP-1 |
| N-DDD2-Fab-hMN-14 | CEACAM5 |

### Example 8

### Sequence Variants for DNL

In certain preferred embodiments, the AD and DDD sequences incorporated into the DNL construct comprise the amino acid sequences of AD1, AD2, AD3, DDD1, DDD2, DDD3 or DDD3C as discussed above. However, in alternative embodiments sequence variants of AD and/or DDD moieties may be utilized in construction of the DNL complexes. For example, there are only four variants of human PKA DDD sequences, corresponding to the DDD moieties of PKA RIα, RIIα, RIβ and RIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 54)
SLRECELYVQKHNIQALLKDVSIVQLCTARPERPMAFLREYFEKLEKEEAK
PKA RIβ
(SEQ ID NO: 55)
SLKGCELYVQLHGIQQVLKDCIVHLCISKPERPMKFLREHFEKLEKEENRQILA
PKA RIIα
(SEQ ID NO: 56)
SHIQIPPGLTELLQGYTVEVGQQPPDLVDFAVEYFTRLREARRQ
PKA RIIβ
(SEQ ID NO: 57)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Can et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

For example, Kinderman et al. (2006) examined the crystal structure of the AD-DDD binding interaction and concluded that the human DDD sequence contained a number of conserved amino acid residues that were important in either dimer formation or AKAP binding, underlined in SEQ ID NO:45 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO: 45)
SHIQIPPGLTELLQGYTVEVLRQQPPDLVEFAVEYFTRLREARA

Alto et al. (2003) performed a bioinformatic analysis of the AD sequence of various AKAP proteins to design an RII selective AD sequence called AKAP-IS (SEQ ID NO:47), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:47. The skilled artisan will realize that in designing sequence variants of the AD sequence, one would desirably avoid changing any of the underlined residues, while conservative amino acid substitutions might be made for residues that are less critical for DDD binding.

AKAP-IS
(SEQ ID NO: 47)
QIEYLAKQIVDNAIQQA

Gold (2006) utilized crystallography and peptide screening to develop a SuperAKAP-IS sequence (SEQ ID NO:58), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of Mkt. In this sequence, the N-terminal Q residue is numbered as residue number 4 and the C-terminal A residue is residue number 20. Residues where substitutions could be made to affect the affinity for RIIα were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL constructs. Other alternative sequences that might be substituted for the AKAP-IS AD sequence are shown in SEQ ID NO:59-61. Substitutions relative to the AKAP-IS sequence are underlined. It is anticipated that, as with the AD2 sequence shown in SEQ ID NO:58, the AD moiety may also include the additional N-terminal residues cysteine and glycine and C-terminal residues glycine and cysteine.

SuperAKAP-IS
(SEQ ID NO: 58)
QIEYVAKQIVDYAIHQA
Alternative AKAP sequences
(SEQ ID NO: 59)
QIEYKAKQIVDHAIHQA
(SEQ ID NO: 60)
QIEYHAKQIVDHAIHQA
(SEQ ID NO: 61)
QIEYVAKQIVDHAIHQA

FIG. 2 of Gold et al. disclosed additional DDD-binding sequences from a variety of AKAP proteins, shown below.

RII-Specific AKAPs
AKAP-KL
(SEQ ID NO: 62)
PLEYQAGLLVQNAIQQAI
AKAP79
(SEQ ID NO: 63)
LLIETASSLVKNAIQLSI
AKAP-Lbc
(SEQ ID NO: 64)
LIEEAASRIVDAVIEQVK
RI-Specific AKAPs
AKAPce
(SEQ ID NO: 65)
ALYQFADRFSELVISEAL
RIAD
(SEQ ID NO: 66)
LEQVANQLADQIIKEAT
PV38
(SEQ ID NO: 67)
FEELAWKIAKMIWSDVF
Dual-Specificity AKAPs
AKAP7
(SEQ ID NO: 68)
ELVRLSKRLVENAVLKAV
MAP2D
(SEQ ID NO: 69)
TAEEVSARIVQVVTAEAV
DAKAP1
(SEQ ID NO: 70)
QIKQAAFQLISQVILEAT
DAKAP2
(SEQ ID NO: 71)
LAWKIAKMIVSDVMQQ

Stokka et al. (2006) also developed peptide competitors of AKAP binding to PKA, shown in SEQ ID NO:72-74. The peptide antagonists were designated as Ht31 (SEQ ID NO:72), RIAD (SEQ ID NO:73) and PV-38 (SEQ ID NO:74). The Ht-31 peptide exhibited a greater affinity for the RII isoform of PKA, while the RIAD and PV-38 showed higher affinity for RI.

Ht31
(SEQ ID NO: 72)
DLIEEAASRIVDAVIEQVKAAGAY
RIAD
(SEQ ID NO: 73)
LEQYANQLADQIIKEATE
PV-38
(SEQ ID NO: 74)
FEELAWKIAKMIWSDVFQQC

Hundsrucker et al. (2006) developed still other peptide competitors for AKAP binding to PKA, with a binding constant as low as 0.4 nM to the DDD of the RII form of PKA. The sequences of various AKAP antagonistic peptides are provided in Table 1 of Hundsrucker et al., reproduced in Table 5 below. AKAPIS represents a synthetic RII subunit-binding peptide. All other peptides are derived from the RII-binding domains of the indicated AKAPs.

TABLE 5
AKAP Peptide sequences

| | Peptide Sequence |
|---|---|
| AKAPIS | QIEYLAKQIVDNAIQQA (SEQ ID NO: 47) |
| AKAPIS-P | QIEYLAKQIPDNAIQQA (SEQ ID NO: 75) |
| Ht31 | KGADLIEEAASRIVDAVIEQVKAAG (SEQ ID NO: 76) |
| Ht31-P | KGADLIEEAASRIPDAPIEQVKAAG (SEQ ID NO: 77) |
| AKAP7δ-wt-pep | PEDAELVRLSKRLVENAVLKAVQQY (SEQ ID NO: 78) |
| AKAP7δ-L304T-pep | PEDAELVRTSKRLVENAVLKAVQQY (SEQ ID NO: 79) |
| AKAP7δ-L308D-pep | PEDAELVRLSKRDVENAVLKAVQQY (SEQ ID NO: 80) |
| AKAP7δ-P-pep | PEDAELVRLSKRLPENAVLKAVQQY (SEQ ID NO: 81) |
| AKAP7δ-PP-pep | PEDAELVRLSKRLPENAPLKAVQQY (SEQ ID NO: 82) |
| AKAP7δ-L314E-pep | PEDAELVRLSKRLVENAVEKAVQQY (SEQ ID NO: 83) |
| AKAP1-pep | EEGLDRNEEIKRAAFQIISQVISEA (SEQ ID NO: 84) |
| AKAP2-pep | LVDDPLEYQAGLLVQNAIQQAIAEQ (SEQ ID NO: 85) |
| AKAP5-pep | QYETLLIETASSLVKNAIQLSIEQL (SEQ ID NO: 86) |
| AKAP9-pep | LEKQYQEQLEEEVAKVIVSMSIAFA (SEQ ID NO: 87) |
| AKAP10-pep | NTDEAQEELAWKIAKMIVSDIMQQA (SEQ ID NO: 88) |
| AKAP11-pep | VNLDKKAVLAEKIVAEAIEKAEREL (SEQ ID NO: 89) |
| AKAP12-pep | NGILELETKSSKLVQNIIQTAVDQF (SEQ ID NO: 90) |
| AKAP14-pep | TQDKNYEDELTQVALALVEDVINYA (SEQ ID NO: 91) |
| Rab32-pep | ETSAKDNINIEEAARFLVEKILVNH (SEQ ID NO: 92) |

Residues that were highly conserved among the AD domains of different AKAP proteins are indicated below by underlining with reference to the AKAP IS sequence (SEQ ID NO:47). The residues are the same as observed by Alto et al. (2003), with the addition of the C-terminal alanine residue. (See FIG. 4 of Hundsrucker et al. (2006), incorporated herein by reference.) The sequences of peptide antagonists with particularly high affinities for the RII DDD sequence were those of AKAP-IS, AKAP7δ-wt-pep, AKAP7δ-L304T-pep and AKAP7δ-L308D-pep.

AKAP-IS
(SEQ ID NO: 47)
QIEYLAKQIVDNAIQQA

Carr et al. (2001) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:45. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

(SEQ ID NO: 45)
SHIQIPPGLTELLQGYTVEVLRQQPPDLVEFAVEYFTRLREARA

The skilled artisan will realize that these and other amino acid substitutions in the antibody moiety or linker portions of the DNL constructs may be utilized to enhance the therapeutic and/or pharmacokinetic properties of the resulting DNL constructs.

### Example 9

### Antibody-Dendrimer DNL Complex for siRNA

Cationic polymers, such as polylysine, polyethylenimine, or polyamidoamine (PAMAM)-based dendrimers, form complexes with nucleic acids. However, their potential applications as non-viral vectors for delivering therapeutic genes or siRNAs remain a challenge. One approach to improve selectivity and potency of a dendrimeric nanoparticle may be achieved by conjugation with an antibody that internalizes upon binding to target cells.

We synthesized and characterized a novel immunoconjugate, designated E1-G5/2, which was made by the DNL method to comprise half of a generation 5 (G5) PAMAM dendrimer (G5/2) site-specifically linked to a stabilized dimer of Fab derived from hRS7, a humanized antibody that is rapidly internalized upon binding to the Trop-2 antigen expressed on various solid cancers.

Methods

E1-G5/2 was prepared by combining two self-assembling modules, AD2-G5/2 and hRS7-Fab-DDD2, under mild redox conditions, followed by purification on a Protein L column. To make AD2-G5/2, we derivatized the AD2 peptide with a maleimide group to react with the single thiol generated from reducing a G5 PAMAM with a cystamine core and used reversed-phase HPLC to isolate AD2-G5/2. We produced hRS7-Fab-DDD2 as a fusion protein in myeloma cells, as described in the Examples above.

The molecular size, purity and composition of E1-G5/2 were analyzed by size-exclusion HPLC, SDS-PAGE, and Western blotting. The biological functions of E1-G5/2 were assessed by binding to an anti-idiotype antibody against hRS7, a gel retardation assay, and a DNase protection assay.

Results

E1-G5/2 was shown by size-exclusion HPLC to consist of a major peak (>90%) flanked by several minor peaks. The three constituents of E1-G5/2 (Fd-DDD2, the light chain, and AD2-G5/2) were detected by reducing SDS-PAGE and confirmed by Western blotting. Anti-idiotype binding analysis revealed E1-G5/2 contained a population of antibody-dendrimer conjugates of different size, all of which were capable of recognizing the anti-idiotype antibody, thus suggesting structural variability in the size of the purchased G5 dendrimer. Gel retardation assays showed E1-G5/2 was able to maximally condense plasmid DNA at a charge ratio of 6:1 (+/−), with the resulting dendriplexes completely protecting the complexed DNA from degradation by DNase I.

Conclusion

The DNL technique can be used to build dendrimer-based nanoparticles that are targetable with antibodies. Such agents have improved properties as carriers of drugs, plasmids or siRNAs for applications in vitro and in vivo.

### Example 10

### Maleimide AD2 Conjugate for DNL Dendrimers

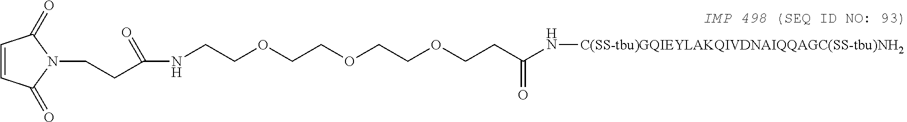

The peptide IMP 498 up to and including the PEG moiety was synthesized on a Protein Technologies PS3 peptide synthesizer by the Fmoc method on Sieber Amide resin (0.1 mmol scale). The maleimide was added manually by mixing the β-maleimidopropionic acid NHS ester with diisopropylethylamine and DMF with the resin for 4 hr. The peptide was cleaved from the resin with 15 mL TFA, 0.5 mL H₂O, 0.5 mL triisopropylsilane, and 0.5 mL thioanisole for 3 hr at room temperature. The peptide was purified by reverse phase HPLC using H₂O/CH₃CN TFA buffers to obtain about 90 mg of purified product after lyophilization.

Synthesis of Reduced G5 Dendrimer (G5/2)

The G-5 dendrimer (10% in MeOH, Dendritic Nanotechnologies), 2.03 g, 7.03×10⁻⁶ mol was reduced with 0.1426 TCEP.HCl 1:1 MeOH/H₂O (=4 mL) and stirred overnight at room temperature. The reaction mixture was purified by reverse phase HPLC on a C-18 column eluted with 0.1% TFA H₂O/CH₃CN buffers to obtain 0.0633 g of the desired product after lyophilization.

Synthesis of G5/2 Dendrimer-AD2 Conjugate

The G5/2 Dendrimer, 0.0469 g (3.35×10⁻⁶ mol) was mixed with 0.0124g of IMP 498 (4.4×10⁻⁶ mol) and dissolved in 1:1 MeOH/1M NaHCO₃ and mixed for 19 hr at room temperature followed by treatment with 0.0751 g dithiothreitol and 0.0441 g TCEP HCl. The solution was mixed overnight at room temperature and purified on a C4 reverse phase HPLC column using 0.1% TFA H₂O/CH₃CN buffers to obtain 0.0033 g of material containing the conjugated AD2 and dendrimer as judged by gel electrophoresis and Western blot.

### Example 11

### Targeted Delivery of siRNA Using Protamine Linked Antibodies

Summary

RNA interference (RNAi) has been shown to down-regulate the expression of various proteins such as HER2, VEGF, Raf-1, bcl-2, EGFR and numerous others in preclinical studies. Despite the potential of RNAi to silence specific genes, the full therapeutic potential of RNAi remains to be realized due to the lack of an effective delivery system to target cells in vivo.

To address this critical need, we developed novel DNL constructs having multiple copies of human protamine tethered to a tumor-targeting, internalizing hRS7 (anti-Trop-2) antibody for targeted delivery of siRNAs in vivo. A DDD2-L-thP1 module comprising truncated human protamine (thP1, residues 8 to 29 of human protamine 1) was produced, in which the sequences of DDD2 and thP1 were fused respectively to the N- and C-terminal ends of a humanized antibody light chain (not shown). The sequence of the truncated hP1 (thP1) is shown below. Reaction of DDD2-L-thP1 with the antibody hRS7-IgG-AD2 under mild redox conditions, as described in the Examples above, resulted in the formation of an E1-L-thP1 complex (not shown), comprising four copies of thP1 attached to the carboxyl termini of the hRS7 heavy chains.

tHP1
(SEQ ID NO: 94)
RSQSRSRYYRQRQRSRRRRRRS

The purity and molecular integrity of E1-L-thP1 following Protein A purification were determined by size-exclusion HPLC and SDS-PAGE (not shown). In addition, the ability of E1-L-thP1 to bind plasmid DNA or siRNA was demonstrated by the gel shift assay (not shown). E1-L-thP1 was effective at binding short double-stranded oligonucleotides (not shown) and in protecting bound DNA from digestion by nucleases added to the sample or present in serum (not shown).

The ability of the E1-L-thP1 construct to internalize siRNAs into Trop-2-expressing cancer cells was confirmed by fluorescence microscopy using FITC-conjugated siRNA and the human Calu-3 lung cancer cell line (not shown).

Methods

The DNL technique was employed to generate E1-L-thP1. The hRS7 IgG-AD module, constructed as described in the Examples above, was expressed in myeloma cells and purified from the culture supernatant using Protein A affinity chromatography. The DDD2-L-thP1 module was expressed as a fusion protein in myeloma cells and was purified by Protein L affinity chromatography. Since the CH3-AD2-IgG module possesses two AD2 peptides and each can bind to a DDD2 dimer, with each DDD2 monomer attached to a protamine moiety, the resulting E1-L-thP1 conjugate comprises four protamine groups. E1-L-thp1 was formed in nearly quantitative yield from the constituent modules and was purified to near homogeneity (not shown) with Protein A.

DDD2-L-thP1 was purified using Protein L affinity chromatography and assessed by size exclusion HPLC analysis and SDS-PAGE under reducing and nonreducing conditions (data not shown). A major peak was observed at 9.6 min (not shown). SDS-PAGE showed a major band between 30 and 40 kDa in reducing gel and a major band about 60 kDa (indicating a dimeric form of DDD2-L-thP1) in nonreducing gel (not shown). The results of Western blotting confirmed the presence of monomeric DDD2-L-tP1 and dimeric DDD2-L-tP1 on probing with anti-DDD antibodies (not shown).

To prepare the E1-L-thP1, hRS7-IgG-AD2 and DDD2-L-thP1 were combined in approximately equal amounts and reduced glutathione (final concentration 1 mM) was added. Following an overnight incubation at room temperature, oxidized glutathione was added (final concentration 2 mM) and the incubation continued for another 24 h. E1-L-thP1 was purified from the reaction mixture by Protein A column chromatography and eluted with 0.1 M sodium citrate buffer (pH 3.5). The product peak was neutralized, concentrated, dialyzed with PBS, filtered, and stored in PBS containing 5% glycerol at 2 to 8° C. The composition of E1-L-thP1 was confirmed by reducing SDS-PAGE (not shown), which showed the presence of all three constituents (AD2-appended heavy chain, DDD2-L-htP1, and light chain).

The ability of DDD2-L-thP1 (not shown) and E1-L-thP1 (not shown) to bind DNA was evaluated by gel shift assay. DDD2-L-thP1 retarded the mobility of 500 ng of a linear form of 3-kb DNA fragment in 1% agarose at a molar ratio of 6 or higher (not shown). E1-L-thP1 retarded the mobility of 250 ng of a linear 200-bp DNA duplex in 2% agarose at a molar ratio of 4 or higher (not shown), whereas no such effect was observed for hRS7-IgG-AD2 alone (not shown). The ability of E1-L-thP1 to protect bound DNA from degradation by exogenous DNase and serum nucleases was also demonstrated (not shown).

The ability of E1-L-thP1 to promote internalization of bound siRNA was examined in the Trop-2 expressing ME-180 cervical cell line (not shown). Internalization of the E1-L-thP1 complex was monitored using FITC conjugated goat anti-human antibodies. The cells alone showed no fluorescence (not shown). Addition of FITC-labeled siRNA alone resulted in minimal internalization of the siRNA (FIG. 3, upper right). Internalization of E1-L-thP1 alone was observed in 60 minutes at 37° C. (not shown). E1-L-thP1 was able to effectively promote internalization of bound FITC-conjugated siRNA (not shown). E1-L-thP1 (10 μg) was mixed with FITC-siRNA (300 nM) and allowed to form E1-L-thP1-siRNA complexes which were then added to Trop-2-expressing Calu-3 cells. After incubation for 4 h at 37° C. the cells were checked for internalization of siRNA by fluorescence microscopy (not shown).

The ability of E1-L-thP1 to induce apoptosis by internalization of siRNA was examined. E1-L-thP1 (10 μg) was mixed with varying amounts of siRNA (AllStars Cell Death siRNA, Qiagen, Valencia, Calif.). The E1-L-thP1-siRNA complex was added to ME-180 cells. After 72 h of incubation, cells were trypsinized and annexin V staining was performed to evaluate apoptosis. The Cell Death siRNA alone or E1-L-thP1 alone had no effect on apoptosis (not shown). Addition of increasing amounts of E1-L-thP1-siRNA produced a dose-dependent increase in apoptosis (not shown). These results show that E1-L-thP1 could effectively deliver siRNA molecules into the cells and induce apoptosis of target cells.

Conclusions

The DNL technology provides a modular approach to efficiently tether multiple protamine molecules to the anti-Trop-2 hRS7 antibody resulting in the novel molecule E1-L-thP1. SDS-PAGE demonstrated the homogeneity and purity of E1-L-thP1. DNase protection and gel shift assays showed the DNA binding activity of E1-L-thP1. E1-L-thP1 internalized in the cells like the parental hRS7 antibody and was able to effectively internalize siRNA molecules into Trop-2-expressing cells, such as ME-180 and Calu-3.

The skilled artisan will realize that the DNL technique is not limited to any specific antibody or siRNA species. Rather, the same methods and compositions demonstrated herein can be used to make targeted delivery complexes comprising any antibody, any siRNA carrier and any siRNA species. The use of a bivalent IgG in targeted delivery complexes would result in prolonged circulating half-life and higher binding avidity to target cells, resulting in increased uptake and improved efficacy.

### Example 12

### Apoptosis of Pancreatic Cancer Using siRNAs Against CD74 and CEACAM6

The siRNAs for CD74 (sc-35023, Santa Cruz Biotechnology, Santa Cruz, Calif.) and CEACAM6 [sense strand 5′-CCGGACAGUUCCAUGUAUAdTdT-3′ (SEQ ID NO:95)], are obtained from commercial sources. Sense and antisense siRNAs are dissolved in 30 mM HEPES buffer to a final concentration of 20 μM, heated to 90° C. for 1 min and incubated at 37° C. for 60 min to form duplex siRNA. The duplex siRNA is mixed with E1-L-thP1 and incubated with BxPC-3 (CEACAM6-siRNA) and Capan2 (CD74-siRNA) cells. After 24 h, the changes in the levels of mRNA for the corresponding proteins are determined by real time quantitative PCR analysis. The levels CD74 and CEACAM6 proteins are determined by Western blot analysis and immunohistochemistry. Controls include nonspecific siRNA and the non-targeting DNL complex 20-L-thPl, which contains a humanized anti-CD20 antibody (hA20).

The effects of reduced expression of CD74 and CEACAM6 on the growth of pancreatic cancer cells is determined using the clonogenic assay. About 1×10³BxPC-3 cells are plated and treated with E1-L-thP1 carrying CEACAM6-siRNA. Media is changed every 3-4 days and after 14 days colonies are fixed with 4% para-formaldehyde solution, stained with 0.5% trypan blue and counted. Similar experiments are performed for Capan2 cells using E1-L-thP1 carrying CD74-siRNA. The effect of E1-L-thP1 carrying both CEACAM6- and CD74-siRNAs on inhibiting the growth of BxPC-3 and Capan2 cells is determined. Cell proliferation by the MTS assay is performed.

Two xenograft models are established in female athymic nu/nu mice (5 weeks of age, weighing 18-20 g). The subcutaneous model has BxPC-3 (ATCC No. CRL-1687) and Capan2 (ATCC No. HTB-80) implanted in opposite flanks of each animal with treatment initiated once tumors reach 50 mm³. The orthotopic model bears only BxPC-3 cells and treatment is started 2 weeks after implantation.

For the subcutaneous model, the efficacy of E1-L-thP1 to deliver a mixture of CEACAM6- and CD74-siRNAs is assessed and compared to that of E1-L-thP1 to deliver CEACAM6-, CD74-, or control siRNA individually. Additional controls are saline and the use of 20-L-thP1 instead of E1-L-thP1 to deliver the specific and control siRNAs. The dosage, schedule, and administration are 150 μg/kg based on siRNA, twice weekly for 6 weeks, and via tail vein injection (Table 6). Cells are expanded in tissue culture, harvested with Trypsin/EDTA, and re-suspended with matrigel (1:1) to deliver 5×10⁶ cells in 300 μL.

Animals are monitored daily for signs of toxicity and weighed twice weekly. Tumor dimensions are measured weekly and tumor volumes calculated.

The orthotopic model is set up as follows. Briefly, nude mice are anesthetized and a left lateral abdominal incision is made. The spleen and attached pancreas are exteriorized with forceps. Then 50 μL of a BxPC-3 cell suspension (2×10⁶ cells) is injected into the pancreas. The spleen and pancreas are placed back into the abdominal cavity and the incision closed. Therapy begins two weeks after implantation. Mice are treated systemically with CEACAM6- or control siRNA bound to E1-L-thP1 or 20-L-thP1 with the same dosing schedule and route as the subcutaneous model. Animals are monitored daily and weighed weekly.

TABLE 6
Subcutaneous model with dual tumors

| | | | |
|---|---|---|---|
| Group | (N) | Treatment | Dose/Schedule |

Specific Therapy

| | | | |
|---|---|---|---|
| 1 | 12 | E1-L-thP1-CEACAM6- | 150 μg/kg i.v. |
| | | siRNA | (twice weekly × 6) |
| 2 | 12 | E1-L-thP1-CD74-siRNA | 150 μg/kg i.v. |
| | | | (twice weekly × 6) |
| 3 | 12 | E1-L-thP1-CEACAM6- | 150 μg/kg each i.v. |
| | | siRNA + | (twice weekly × 6) |
| | | E1-L-thP1-CD74-siRNA | |

Controls

| | | | |
|---|---|---|---|
| 4 | 12 | Saline | 100 μL i.v. |
| | | | (twice weekly × 6) |
| 5 | 12 | 20-L-thP1-CEACAM6- | 150 μg/kg i.v. |
| | | siRNA | (twice weekly × 6) |
| 6 | 12 | 20-L-thP1-CD74-siRNA | 150 μg/kg i.v. |
| | | | (twice weekly × 6) |
| 7 | 12 | E1-L-thP1-control-siRNA | 150 μg/kg each i.v. |
| | | | (twice weekly × 6) |
| 8 | 12 | 20-L-thP1-control-siRNA | 150 μg/kg each i.v. |
| | | | (twice weekly × 6) |

The results of the study show that both CEACAM6 and CD74 siRNA are internalized into pancreatic cancer cells by the E1-L-thP1 DNL construct and induce apoptosis of pancreatic cancer, while the control DNL construct with non-targeting anti-CD20 antibody is ineffective to induce siRNA uptake or cancer cell death.

It will be readily apparent to one skilled in the art that varying substitutions and modifications may be made to the invention disclosed herein without departing from the scope and spirit of the invention. Thus, such additional embodiments are within the scope of the present invention.

What is claimed is:

1. A method of killing a cell that is resistant to anti-CD74 antibody comprising
a. exposing the anti-CD74 resistant cell to interferon-γ;
b. increasing expression of CD74 on the cell surface; and
c. exposing the cell to an anti-CD74 antibody or antigen-binding fragment thereof after the cell has been exposed to interferon-γ,
wherein exposing the cell to interferon-γ increases the sensitivity of the cell to the anti-CD74 antibody.

2. The method of claim 1, wherein the cell is a diseased cell.

3. The method of claim 2, wherein the disease is selected from the group consisting of cancer, autoimmune disease and immune dysfunction disease.

4. The method of claim 3, wherein the cancer is selected from the group consisting of hematopoietic cancer, B-cell leukemia, B-cell lymphoma, non-Hodgkin's lymphoma (NHL), multiple myeloma, chronic lymphocytic leukemia, acute lymphocytic leukemia, acute myelogenous leukemia, glioblastoma, follicular lymphoma. diffuse large B cell lymphoma, colon cancer, pancreatic cancer, renal cancer, lung cancer, stomach cancer, breast cancer, prostate cancer, ovarian cancer and melanoma.

5. The method of claim 3, wherein the immune dysregulation disease is graft-versus-host disease or organ transplant rejection.

6. The method of claim 3, wherein the autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

7. The method of claim 1, wherein the anti-CD74 antibody competes for binding to CD74 with, or binds to the same epitope of CD74 as, an antibody comprising the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

8. The method of claim 1, wherein the anti-CD74 antibody comprises the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

9. The method of claim 1, wherein the anti-CD74 antibody or fragment thereof is a naked antibody or fragment thereof.

10. The method of claim 9, further comprising exposing the cell to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

11. The method of claim 1, wherein the anti-CD74 antibody or fragment thereof is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

12. The method of claim 11, wherein the anti-CD74 antibody or fragment thereof is conjugated to a second antibody or fragment thereof to form a bispecific antibody.

13. The method of claim 12, wherein the second antibody or fragment thereof binds to an antigen selected from the group consisting of carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CXCR4, CXCR7, CXCL12, HIF-1α, AFP, PSMA, CEACAM5, CEACAM6, B7, ED-B of fibronectin, Factor H, FHL-1, Flt-3, folate receptor, GROB, HMGB-1, hypoxia inducible factor (HIF), HM1.24, insulin-like growth factor-1 (IGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, MAGE, mCRP, MCP-1, MIP-1A, MIP-1B, MIF, MUC1, MUC2, MUC3, MUC4, MUC5, NCA-95, NCA-90, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, P1GF, complement factors C3, C3a, C3b, C5a, C5, and an oncogene product.

14. The method of claim 12, wherein the bispecific antibody is a dock-and-lock complex.

15. The method of claim 11, wherein the therapeutic agent is selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

16. The method of claim 11, wherein the therapeutic agent is a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ^{20I}Tl, ²⁰³Hg, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

17. The method of claim 11, wherein the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

18. The method of claim 11, wherein the therapeutic agent is an immunomodulator selected from the group consisting of erythropoietin, thrombopoietin tumor necrosis factor-α (TNF), TNF-β, granulocyte-colony stimulating factor (G-CSF), granulocyte macrophage-colony stimulating factor (GM-CSF), interferon-α, interferon-β, interferon-γ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin and LT.

19. The method of claim 1, wherein the antibody fragment is selected from the group consisting of F(ab′)₂, F(ab)₂, Fab′, Fab, Fv, scFv and single domain antibody.

20. The method of claim 1, wherein the anti-CD74 antibody is a chimeric, humanized or human anti-CD74 antibody.
